(19) United States
(12) Patent Application Publication
(10) Pub. No.: US 2026/0269069 A1
(43) Pub. Date: Sep. 10, 2026

(54) USER INTERFACE FRAMEWORK FOR CAPTURING HEALTH INFORMATION

(71) Applicant: Hedia ApS, Copenhagen (DK)

(72) Inventors: Mads Obdrup Jakobsen, København (DK); Jonas Meinertz Hansen, Hillerød (DK); Gkikas Karagkounis, Frederiksberg (DK); Astrid Karnøe Knudsen; Anders Patrick Jørgensen, Humlebaek (DK)

(21) Appl. No.: 19/166,003

(22) PCT Filed: Mar. 6, 2024
(86) PCT No.: PCT/EP2024/055851

§ 371 Date: Sep. 16, 2025

Related U.S. Application Data
(60) Provisional application No. 63/452,963, filed on Mar. 17, 2023.

(51) Int. Cl. G16H 50/30; G16H 20/17; G16H 50/50; G16H 50/70

(52) U.S. Cl. CPC G16H 50/30; G16H 20/17; G16H 50/50; G16H 50/70

(57) ABSTRACT

Apparatuses, systems, and techniques are described to provide a user interface framework for capturing health information. In one or more implementations, diabetes healthcare data can be captured via the user interface framework and analyzed to provide recommendations to individuals for the adjustment of blood glucose levels. The diabetes healthcare data can be analyzed using one or more machine learning techniques to predict blood glucose levels of individuals. The predicted blood glucose levels can be used to determine at least one of an amount of insulin intake or carbohydrate consumption to modify blood glucose levels of individuals.

### BACKGROUND

Diabetes is a biological condition that affects millions of patients worldwide and is characterized by the inability of a patient's body to produce and/or process insulin. Diabetes can result in the inability of patients to regulate their blood glucose levels. Typically, patients adjust blood glucose levels by receiving doses of insulin and/or consuming an amount of carbohydrates to help maintain blood glucose levels in a healthy range. In many instances, patients can monitor blood glucose levels using sensors that indicate current blood glucose levels of patients. Based on the data provided by the sensors, patients can determine when their blood glucose levels need adjustment and act accordingly by consuming an amount of carbohydrates or receiving an insulin dose.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example environment to obtain data and determine a recommendation to adjust a blood glucose level of a patient, according to one or more example implementations.

FIG. 2 illustrates an example framework of a machine learning architecture to determine a prediction of a blood glucose level of a patient, according to one or more example implementations.

FIG. 3 illustrates an example framework that stores and executes software code according to different levels of a regulatory scheme, according to one or more example implementations.

FIG. 4 illustrates an example framework to capture user input and display recommendations to adjust a blood glucose level of a patient, according to one or more example implementations.

FIG. 5 is a flow diagram of an example process to predict a blood glucose level of a patient using a machine learning architecture, according to one or more example implementations.

FIG. 6 is a flow diagram of an example process to determine a recommendation to adjust a blood glucose level of a patient using software code that is maintained and executed in accordance with different levels of a regulatory scheme, according to one or more example implementations.

FIG. 7 illustrates a diagrammatic representation of a machine in the form of a computer system within which a set of instructions may be executed for causing the machine to perform any one or more of the methodologies or processes discussed herein, according to one or more example implementations.

FIG. 8 is a block diagram illustrating an example architecture of software, which can be installed on any one or more of the devices described herein.

### DETAILED DESCRIPTION

Conventional techniques for the collection and analysis of data that can be used to treat individuals in which diabetes is present are mainly directed to gathering real-time blood glucose indicators using one or more sensors. In at least some examples, the sensors can be located under the skin and provide measurements indicating amounts of glucose disposed between cells. In these situations, the sensors can provide estimates of blood glucose levels for individuals. In other situations, sensors can be included in an electronic device and the sensors can analyze blood samples extracted from the individuals. In these scenarios, the blood glucose levels are directly determined by the glucose sensors. In some cases, the blood samples can be disposed on a plastic strip that is inserted into the electronic device.

Based on the information obtained from the various sensors, individuals can then use the real-time data indicative of blood glucose levels to make decisions about how much insulin to inject and/or an amount of carbohydrates to consume. In some scenarios, the blood glucose indicators can be inaccurate, and individuals can act on the inaccurate blood glucose indicators in a way that negatively impacts the health of the individuals. For examples, individuals can inject amounts of insulin and/or consume amounts of carbohydrates that can cause individuals to experience hypoglycemic events or hyperglycemic events. Additionally, simply relying on real-time blood glucose indicators does not take into account future blood glucose level changes. Therefore, individuals may not be able to take insulin or consume an amount of carbohydrates at a current time to avoid a future spike or trough in blood glucose levels when merely real-time blood glucose indicators are known.

The systems, processes, techniques, and methods described herein are different from conventional systems that monitor real-time blood glucose indicators of individuals. In particular, the systems described herein can be implemented to predict future blood glucose levels using one or more machine learning algorithms. In addition, implementations described herein can inform individuals when an accuracy of real-time blood glucose indicators and/or predicted blood glucose levels is relatively low. Further, the implementations described herein can analyze, in an automated manner, not only blood glucose indicators, but also lifestyle factors to accurately determine amounts of insulin to take and/or amounts of carbohydrates to consume to maintain blood glucose levels in a healthy range. Additionally, the implementations described herein can include architectures that arrange software code according to regulatory framework levels. For example, first software code that provides first functionality covered under a first regulatory framework level can be stored, maintained, or executed according to a first set of conditions and second software code that provides second functionality covered under a second regulatory framework level can be stored, maintained, or executed according to a second set of conditions different from the first set of conditions. In this way, changes to the first software code and the second software code can be made independently and avoid triggering regulatory requirements that can delay or prevent updates to various features of software code that can be used to manage treatment of diabetes.

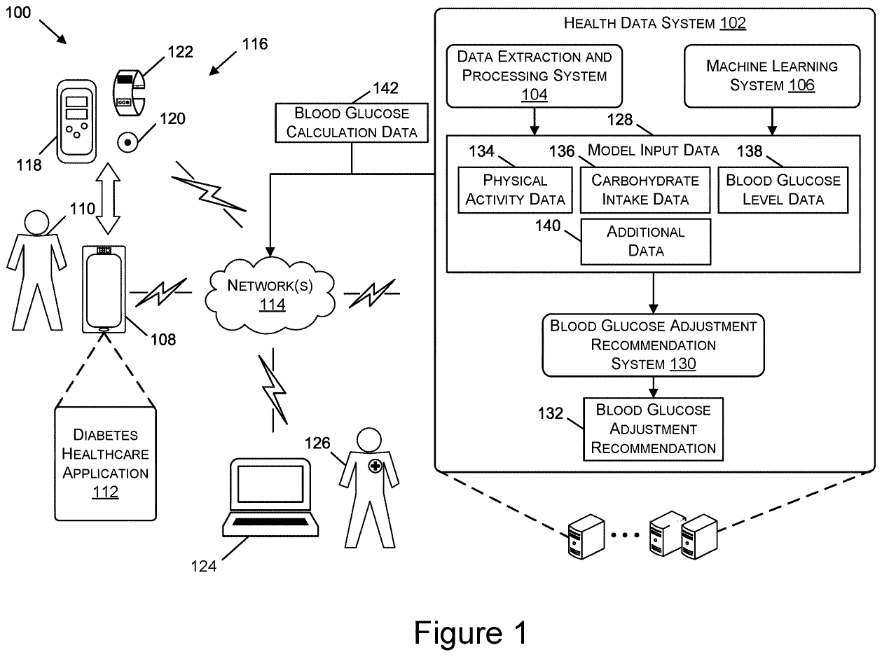

FIG. 1 illustrates an example environment 100 to obtain data used in the detection, diagnosis, and determining candidate treatments of one or more biological conditions or issues. In addition, as used herein, a biological condition can refer to an abnormality of function and/or structure in an individual to such a degree as to produce or threaten to produce a detectable feature of the abnormality. A biological condition can be characterized by external and/or internal characteristics, signs, and/or symptoms that indicate a deviation from a biological norm in one or more populations. In one or more additional examples, a biological condition can include at least one of one or more diseases, one or more disorders, one or more injuries, one or more syndromes, one or more disabilities, one or more infections, one or more isolated symptoms, or other atypical variations of biological structure and/or function of individuals. Additionally, a treatment, as used herein, can refer to a substance, procedure, routine, device, and/or other intervention that can be administered or performed with the intent of treating one or more effects of a biological condition in an individual. In one or more examples, a treatment may include a substance that is metabolized by the individual. The substance may include a composition of matter, such as a pharmaceutical composition. The substance may be delivered to the individual via a number of methods, such as ingestion, injection, absorption, or inhalation. A treatment may also include physical interventions, such as one or more surgeries. In at least some examples, the treatment can include a therapeutically meaningful intervention. In some instances, described herein, a biological condition can be referred to as a “biological issue.”

In one or more illustrative examples, the biological condition can include diabetes. In one or more additional illustrative examples, the biological condition can include at least one of type I Diabetes, type II Diabetes, or a form of pre-diabetes. In various examples, as used herein, diabetes or diabetes-related condition can include at least one of type I Diabetes, type II Diabetes, a form of pre-diabetes, or other biological conditions resulting from diabetes or a diabetes-like condition. Additionally, a treatment, as used herein, can refer to a substance, procedure, routine, device, and/or other intervention that can administered or performed with the intent of alleviating one or more effects of a biological condition in an individual.

The environment 100 can include a health data system 102. The health data system 102 can obtain diabetes healthcare data from one or more sources. The diabetes healthcare data can include measurements of blood glucose levels of individuals. Additionally, the diabetes healthcare data can include data indicating physical activity of individuals and/or dietary information of patients. Further, the diabetes healthcare data can include medical records of individuals. The medical records can include imaging information, laboratory test results, diagnostic test information, clinical observations, dental health information, notes of healthcare practitioners, medical history forms, diagnostic request forms, medical procedure order forms, medical information charts, one or more combinations thereof, and so forth. In various examples, for a given individual, medical records can include information obtained from one or more healthcare practitioners that corresponds to the individual, such as a healthcare practitioner that has treated the individual or a healthcare practitioner that has generated at least a portion of the medical records included in the diabetes healthcare data.

The diabetes healthcare data can be analyzed by the health data system 102 to determine one or more recommendations for treatment of diabetes. For example, the diabetes healthcare data can be analyzed to determine a recommendation that indicates an amount of insulin to be provided to an individual. Additionally, the diabetes healthcare data can be analyzed to determine a recommendation that indicates an amount of carbohydrates to be consumed by an individual. In one or more examples, the health data system 102 can analyze at least one of historical blood glucose measurements or predicted blood glucose measurements to determine a recommendation for an individual. In various implementations, an application executed by computing devices of individuals and/or computing devices of healthcare practitioners can access the health data system 102 to obtain diabetes healthcare data and/or recommendations related to diabetes health of the individuals. In one or more further examples, at least a portion of the functionality of the health data system 102 to generate recommendations for the treatment of diabetes can be performed by one or more applications executed by computing devices of individuals being treated for a diabetes-related condition and/or computing devices of healthcare practitioners.

The health data system 102 can be implemented by one or more computing devices. The one or more computing devices can include one or more server computing devices, one or more desktop computing devices, one or more laptop computing devices, one or more tablet computing devices, one or more mobile computing devices, or combinations thereof. In certain implementations, at least a portion of the one or more computing devices can be implemented in a distributed computing environment. For example, at least a portion of the one or more computing devices can be implemented in a cloud computing architecture.

The health data system 102 can include a data extraction and processing system 104. The data extraction and processing system 104 can obtain data from a number of data sources. The data obtained by the data extraction and processing system 104 can be received from one or more computing devices. In addition, the data obtained by the data extraction and processing system 104 can be extracted from one or more websites. Further, the data obtained by the data extraction and processing system 104 can be retrieved from one or more data stores. At least a portion of the one or more data stores can be located remotely from the health data system 102. In various implementations, the one or more data stores can be maintained by third parties that are different from the one or more entities that implement the health data system 102. The one or more data stores can include one or more databases. The data extraction and processing system 104 can also obtain data from one or more sensors. In one or more illustrative examples, the data extraction and processing system 104 can obtain data from one or more glucose monitoring sensors. In one or more additional illustrative examples, the data extraction and processing system 104 can obtain data from one or more continuous glucose monitors that determine indicators of blood glucose levels. Additionally, the data extraction and processing system 104 can obtain data from one or more blood glucose sensors that directly measure blood glucose levels of subjects based on blood samples obtained from the subjects.

The health data system 102 can also include a machine learning system 106. The machine learning system 106 can implement one or more machine learning techniques to analyze diabetes healthcare data. The machine learning system 106 can analyze the diabetes healthcare data to predict a future blood glucose level of a patient. In one or more illustrative examples, the machine learning system 106 can implement at least one of one or more convolutional neural networks or one or more additional artificial neural networks to analyze diabetes healthcare data of individuals. Additionally, the machine learning system 106 can implement one or more machine learning techniques in relation to one or more dilation rates to analyze diabetes healthcare data of individuals.

The environment 100 can also include a computing device 108 that is operated by an individual 110. The computing device 108 can include a mobile computing device, a smart phone, a tablet computing device, a laptop computing device, a desktop computing device, one or more combinations thereof, and the like. The computing device 108 can send diabetes healthcare data and/or other health information of the individual 110 to the health data system 102. The computing device 108 can also access health information of the individual 110 via the health data system 102. The individual 110 can be an individual that has been previously diagnosed with diabetes. For example, the individual 110 can have previous blood-related information resulting in the individual 110 being diagnosed with diabetes by one or more healthcare practitioners. In one or more additional examples, the individual 110 may be undergoing initial testing for diabetes or the individual 110 may have been previously tested for diabetes with no prior diagnosis of diabetes. In one or more further examples, the individual 110 can be exhibiting pre-diabetic symptoms.

The computing device 108 can transmit and receive data via one or more networks 114. The one or more networks 114 can be representative of any one or combination of multiple different types of wired and/or wireless networks, such as the Internet, cable networks, cellular networks, satellite networks, wide area wireless communication networks, wireless local area networks, wired local area networks, and public switched telephone networks (PSTN).

Diabetes healthcare data of the individual 110 can be obtained by one or more data capture devices 116. The one or more data capture devices 116 can include a blood glucose monitoring device 118, a sensor 120, and a wearable device 122, or one or more combinations thereof. The one or more data capture devices 116 can be in communication with the computing device 108. For example, the one or more of the data capture devices 116 can be in wireless communication with the computing device 108. In one or more illustrative examples, the one or more of the data capture devices 116 can be in communication with the computing device 108 via a Bluetooth network, via a wide area wireless communications network, and/or via a wireless local area network. Additionally, the one or more of the data capture devices 116 can be in communication with the computing device 108 via a wired connection between the one or more data capture devices 116 and the computing device 108. To illustrate, the one or more of the data capture devices 116 can be coupled to the computing device 108 using a Universal Serial Bus (USB) interface and/or a micro-USB interface. Although the one or more data capture devices 116 are shown in the illustrative example of FIG. 1 to be separate from the computing device 108, in various implementations, one or more components of the data capture devices 116 can be included in the computing device 108.

The blood glucose monitoring device 118 can include one or more components to collect blood glucose data. In one or more examples, the blood glucose monitoring device 118 can be in communication with the sensor 120. The sensor 120 can include circuitry that is configured to detect indicators of blood glucose disposed in fluids located beneath the skin of the individual. The sensor 120 can transmit signals to the blood glucose monitoring device 118 that correspond to indicators of blood glucose measurements for the individual 110. In various examples, the sensor 120 can be embedded below the skin of the individual 110 and wirelessly transmit signals to the blood glucose monitoring device 118, such as using one or more Bluetooth communication protocols or one or more additional wireless communication protocols (e.g., one or more IEEE 802.11 wireless communication protocols). Additionally, at least one of the blood glucose monitoring device 118 or the sensor 120 can transmit blood glucose data and/or indicators of blood glucose data to the computing device 108. In one or more further examples, the blood glucose monitoring device 118 can include one or more ports to accept strips that include a blood sample obtained from the individual 110. In these scenarios, the blood glucose monitoring device 118 can include circuitry that analyzes the blood sample and detects blood glucose levels in the blood sample. The blood glucose monitoring device 118 can include a display device that displays a current blood glucose measurement. In at least some examples, the blood glucose monitoring device 118 can display historical blood glucose measurements.

The wearable device 122 can be worn by the individual 110 and can obtain health information about the individual 110. The wearable device 122 can include a watch, glasses, a ring, an anklet, jewelry, or another item that can attach to a body part. In addition, the wearable device 122 can include one or more sensors that detect one or more physiological characteristics of the individual 110. In one or more examples, the wearable device 122 can include at least one of circuitry or one or more mechanical components to detect heart rate, blood pressure, body temperature, blood oxygen level, one or more combinations thereof, and the like.

In various implementations, the computing device 108 can execute one or more applications to communicate with the health data system 102. In one or more examples, the computing device 108 can execute an instance of a diabetes healthcare application 112. The diabetes healthcare application 112 can collect diabetes healthcare data and send the diabetes healthcare data to the health data system 102. In at least some examples, the diabetes healthcare application 112 can cause one or more user interfaces to be displayed that include user interface elements to capture diabetes healthcare data. For example, the diabetes healthcare application 112 can cause one or more user interfaces to be displayed that include one or more user interface elements to capture dietary information related to the individual 110. To illustrate, the diabetes healthcare application 112 can cause one or more user interfaces to be displayed to capture carbohydrate consumption of the individual 110. In one or more illustrative examples, the diabetes healthcare application 112 can provide user interface features to log amounts of carbohydrates consumed at a given time, to log types of foods consumed at a given time, to indicate food and/or drink consumed by the individual 110 as part of a snack and/or meal, or one or more combinations thereof. In one or more additional examples, the diabetes healthcare application 112 can provide user interface features to log physical activity data. In one or more further examples, the diabetes healthcare application can provide user interface features to log insulin intake by the individual 110. The insulin intake information can indicate a time and an amount of a dose of insulin received by the individual 110.

Additionally, the diabetes healthcare application 112 can provide one or more notifications that are accessible by the individual 110. For example, the diabetes healthcare application 112 can generate one or more notifications that can indicate blood glucose levels of the individual 110. To illustrate, the diabetes healthcare application 112 can generate one or more notifications that indicate a current blood glucose level of the individual 110. The diabetes healthcare application 112 can also generate one or more notifications that indicate a predicted blood glucose level of the individual 110 at one or more future times. Further, the diabetes healthcare application 112 can generate one or more notifications indicating that a blood glucose level of the individual 110 is outside of a specified range of blood glucose levels. In one or more illustrative examples, the diabetes healthcare application 112 can generate one or more notifications indicating that at least one a current blood glucose level or a predicted blood glucose level of the individual 110 is less than a lower threshold blood glucose level or greater than an upper threshold blood glucose level. In one or more additional examples, the diabetes healthcare application 112 can generate one or more notifications indicating that at least one of a current blood glucose level or a predicted blood glucose level may not have an expected level of accuracy. In one or more further examples, the diabetes healthcare application 112 can generate one or more notifications indicating a recommendation for at least one of an amount of insulin to take or an amount of carbohydrates to consume to modify a blood glucose level of the individual 110.

In at least some examples, the diabetes healthcare application 112 can include user interface features that enable access to profile data of the individual 110 and access to one or more settings of the diabetes healthcare application 112. The one or more settings of the diabetes healthcare application 112 can be related to notifications generated by the diabetes healthcare application 112. The one or more settings of the diabetes healthcare application 112 can also correspond to adjustments for at least one of insulin intake or carbohydrate consumption for the individual 112 according to at least one of blood glucose levels, blood ketone levels, physical activity, or dietary information.

The environment 100 can also include an additional computing device 124 that is operated by a healthcare practitioner 126. The additional computing device 124 can include a mobile computing device, a smart phone, a tablet computing device, a laptop computing device, a desktop computing device, one or more combinations thereof, and the like. The individual 110 can be a patient of the healthcare practitioner 126 and the healthcare practitioner 126 can provide at least one of a treatment, a treatment protocol, or medical advice to the individual 110 for one or more biological conditions of the individual 110. In one or more implementations, the healthcare practitioner 126 can provide at least one of a treatment, a treatment protocol, or medical advice to the individual 110 in relation to diabetes. In various examples, the information obtained by the one or more data capture devices 116 corresponding to the individual 110 can be accessed by the healthcare practitioner 126 via the health data system 102 using the additional computing device 124.

The data extraction and processing system 104 can obtain information from a number of data sources. The number of data sources can include at least one of the computing device 108, the one or more data capture devices 116, or the additional computing device 124. The one or more data sources can also include one or more publicly accessible databases and/or one or more privately controlled databases. In at least some examples, the one or more data sources can store information related to at least one of physiological characteristics of individuals in which diabetes is present, demographic characteristics of individuals in which diabetes is present, diagnostic test information for individuals in which diabetes is present, blood glucose levels of individuals in which diabetes is present, insulin intake of individuals in which diabetes is present, carbohydrate consumption of individuals in which diabetes is present, physical activity information for individuals in which diabetes is present, or other health related information for individuals in which diabetes is present.

In various examples, the data extraction and processing system 104 can receive information sent from one or more data sources and store the information in one or more data stores accessible to the health data system 102. Additionally, the data extraction and processing system 104 can obtain information from one or more data sources using one or more queries. The one or more queries can be related to various types of information that can be stored by the one or more data sources. Further, the one or more queries can be associated with one or more keywords. In various implementations, the data extraction and processing system 104 can obtain information from one or more data sources using one or more application programming interface (API) calls. In situations where the data extraction and processing system 104 obtains information from at least one website, the data extraction and processing system 104 can use a web crawler to extract the information from the at least one website.

The data extraction and processing system 104 can process the data obtained from one or more data sources by encoding the data according to one or more formats. For example, the data extraction and processing system 104 can format data obtained from one or more data sources such that the data can be stored and retrieved from a database accessible to the health data system 102. In one or more illustrative examples, the data extraction and processing system 104 can format data obtained from one or more data sources according to one or more data structures. The data extraction and processing system 104 can also process data obtained from one or more data sources such that the data can be analyzed using the one or more machine learning systems 106.

In one or more examples, the data extraction and processing system 104 and the machine learning system 106 can generate model input data 128. The model input data 128 can be accessible to a blood glucose adjustment recommendation system 130 that produces a blood glucose adjustment recommendation 132. In one or more illustrative examples, the blood glucose adjustment recommendation system 130 can implement one or more computational models to determine the blood glucose adjustment recommendation 132. In at least some examples, the blood glucose adjustment recommendation system 130 can include a bolus calculator. In at least some examples, the blood glucose adjustment recommendation system 130 can include one or more components that analyze at least a portion of the model input data 128 to generate the blood glucose adjustment recommendation 132.

The model input data 128 can include physical activity data 134 that includes an amount of physical activity that the individual 110 has performed over a period of time. In one or more examples, the physical activity data 134 can include a number of calories burned over a period of time, intensity level of physical activity, an amount of time that the physical activity was performed, a type of physical activity performed, a time and/or date of the physical activity, a location of the physical activity, one or more combinations thereof, and so forth. In one or more illustrative examples, the physical activity data 134 can correspond to physical activity performed by the individual 110 within a threshold amount of time in relation to a current time. In at least some examples, the physical activity data 134 can include physical activity performed recently by the individual 110. In one or more additional examples, the physical activity data 134 can include historical physical activity information for the individual 110. In one or more further examples, the physical activity data 134 can include physical activity performed by the individual 110 within at least 10 minutes of a current time, within at least 20 minutes of a current time, within at least 30 minutes of a current time, within at least 45 minutes of a current time, within at least 60 minutes of a current time, within at last 90 minutes of a current time, within 120 minutes of a current time, within 3 hours of a current time, within 4 hours of a current time, within 5 hours of a current time, or withing 6 hours of a current time. In various examples, the physical activity data 134 can be obtained from at least one of the wearable device 122 or the computing device 108. In one or more additional examples, the physical activity data 134 can be obtained via one or more user interface elements of the diabetes healthcare application 112 that can capture data corresponding to physical activity performed by the individual 110. In still other examples, the physical activity data 134 can be communicated automatically from at least one of the wearable device 122 or the computing device 108 to the health data system 102 and/or to the diabetes healthcare application 112.

The model input data 128 can also include carbohydrate intake data 136 also referred to herein as carbohydrate consumption data that indicates an amount of carbohydrate consumed by the individual 110 over a period of time. In one or more examples, the carbohydrate intake data 136 can indicate a number of grams of carbohydrates consumed by the individual 110 over a period of time and/or a number of calories attributable to carbohydrates consumed by the individual 110 over the period of time. In at least some examples, the carbohydrate intake data 136 can indicate a type of carbohydrate consumed by the individual 110, such as a simple carbohydrate or a complex carbohydrate. The carbohydrate intake data 136 can also indicate that an amount of carbohydrate was consumed as part of a liquid and/or as part of a solid food. Further, the carbohydrate intake data 136 can indicate at least one of additional food, additional drink, or amounts of protein and/or amounts of fat consumed in conjunction with the amount of carbohydrates. In one or more additional examples, the carbohydrate intake data 136 can indicate a time that an amount of carbohydrates was consumed. In various examples, the carbohydrate intake data 136 can be obtained from at least one of the wearable device 122 or the computing device 108. In one or more further examples, the carbohydrate consumption data 136 can be obtained via one or more user interface elements of the diabetes healthcare application 112 that can capture data corresponding to carbohydrates consumed by the individual 110.

In one or more illustrative examples, the carbohydrate consumption data 136 can correspond to carbohydrates consumed by the individual 110 within a threshold amount of time with respect to a current time. In at least some examples, the carbohydrate consumption data 136 can carbohydrates consumed recently by the individual 110. In one or more additional examples, the carbohydrate consumption data 136 can include historical carbohydrate consumption information for the individual 110. In one or more further examples, the carbohydrate consumption data 136 can one or more amounts of carbohydrates consumed by the individual 110 within at least 10 minutes of a current time, within at least 20 minutes of a current time, within at least 30 minutes of a current time, within at least 45 minutes of a current time, within at least 60 minutes of a current time, within at last 90 minutes of a current time, or within 120 minutes of a current time.

In addition, the model input data 128 can include blood glucose level data 138 that corresponds to indicators of blood glucose levels of the individual 110 over a period of time. In at least some examples, the blood glucose level data 138 can indicate blood glucose levels that are determined periodically. For example, a given amount of time may have elapsed between determining indicators of blood glucose levels included in the blood glucose level data 138. To illustrate, at least 1 minute, at least 2 minutes, at least 3 minutes, at least 4 minutes, at least 5 minutes, at least 6 minutes, at least 8 minutes, at least 10 minutes, at least 12 minutes, or at least 15 minutes may have elapsed between at least a portion of the indicators of blood glucose levels included in the blood glucose level data 138. In one or more examples, the data extraction and processing system 104 can obtain indictors of blood glucose measurements from at least one of the computing device 108, the blood glucose monitoring device, the sensor 120, or the wearable device 122 periodically. In various examples, the data extraction and processing system 104 can obtain indicators of blood glucose measurements from one of more of the data capture devices 116 or the computing device 108 using one or more API calls. In one or more additional examples, the blood glucose level data 138 can be obtained via one or more user interface elements of the diabetes healthcare application 112 that are configured to capture blood glucose level information.

In various examples, the blood glucose level data 138 can include predicted blood glucose levels determined by the machine learning system 106. For example, the machine learning system 106 can implement one or more machine learning techniques to analyze at least one of historical blood glucose levels, historical physical activity information, historical carbohydrate consumption information, recent insulin intake information, or historical blood ketone levels of one or more individuals that includes the individual 110 to determine a prediction of blood glucose levels for the individual 110 at a future time. In this way, the blood glucose adjustment recommendation system 130 can use predicted blood glucose levels generated by the machine learning system 108 to determine the blood glucose adjustment recommendation 132.

In one or more examples the model input data 128 can also include additional data 140 that can be analyzed by the blood glucose adjustment recommendation system 130 to determine the blood glucose adjustment recommendation 132. In various examples, the additional data 140 can include information that has previously been determined, such as in scientific literature, to have an impact on blood glucose levels of individuals. The additional data 140 can also include information that has previously been determined to impact at least one of insulin uptake or insulin production of individuals. In at least some examples, the additional data 140 can include blood ketone levels of the individual 110. The blood ketone levels of the individual 110 can be obtained from at least one of the computing device 108, the blood glucose monitoring device 118, the blood glucose sensor 120, or the wearable device 122. In one or more additional examples, the diabetes healthcare application 112 can include one or more user interface elements that are configured to capture blood ketone levels entered by the individual 110. In one or more further examples, the additional data 140 can include one or more settings stored in conjunction with the diabetes healthcare application 112 that can be used by the blood glucose adjustment recommendation system 130 to determine the blood glucose adjustment recommendation 132.

In still other examples, the additional data 140 can include insulin dose information that indicates one or more periods of time at which the individual 110 received an insulin dose. In these scenarios, the additional data 140 an also indicate an amount of the dose of insulin received by the individual 110. In various examples, the individual 110 can receive the insulin dose via injection. In various examples, the additional data 140 can include a carbohydrate to insulin ratio. The carbohydrate to insulin ratio can be determined by the health data system 102 and can indicate an amount of carbohydrates consumed in relation to an amount of insulin received by the individual 110 over a period of time.

The blood glucose adjustment recommendation 132 can include at least one of a recommended amount of insulin for intake by the individual 110 or a recommended amount of carbohydrate consumption to modify a blood glucose level of the individual 110. In various examples, the blood glucose adjustment recommendation 132 can indicate at least one of a recommended amount of insulin for intake by the individual 110 or a recommended amount of carbohydrate consumption to increase a blood glucose level of the individual 110. In one or more additional examples, the blood glucose adjustment recommendation 132 can indicate at least one of a recommended amount of insulin for intake by the individual 110 or a recommended amount of carbohydrate consumption to decrease a blood glucose level of the individual 110. In one or more further examples, the blood glucose adjustment recommendation 132 can be accessible using the diabetes healthcare application 112.

In various examples, the health data system 102 can provide blood glucose calculation data 142 to one or more devices related to the individual 110. The blood glucose calculation data 142 can include a prediction of blood glucose levels of the individual 110 generated by the machine learning system 106. In one or more illustrative examples, the blood glucose calculation data 142 can include a prediction of blood glucose levels of the individual 110 that are provided to a device related to the individual 110 to determine an additional blood glucose adjustment recommendation for the individual 110. That is, in at least some cases, the health data system 102 can use predicted blood glucose measurements determined by the machine learning system 106 to determine the blood glucose adjustment recommendation 132. In one or more additional examples, the health data system 102 can provide the blood glucose calculation data 142 indicating one or more predicted blood glucose levels of the individual 110 determined by the machine learning system 106 to an application, such as the diabetes healthcare application 112, and/or a glucose monitoring device of the individual 110 that uses the blood glucose calculation data 142 to determine the blood glucose adjustment recommendation for the individual 110.

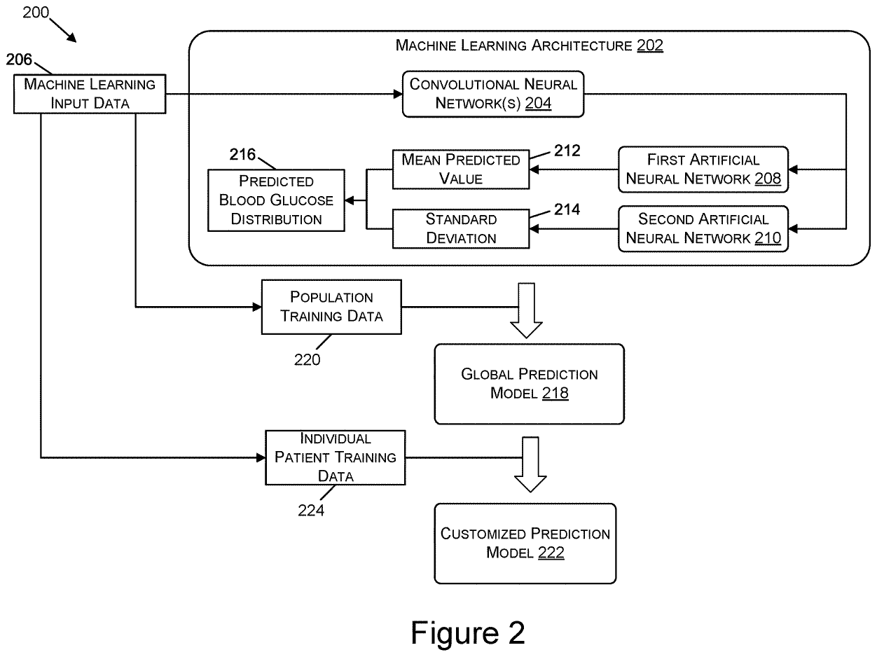

FIG. 2 illustrates an example framework 200 of a machine learning architecture 202 to determine a prediction of a blood glucose level of an individual, according to one or more example implementations. The machine learning architecture 202 can include one or more convolutional neural networks 204. The one or more convolutional neural networks 204 can analyze machine learning input data 206. The machine learning input data 206 can include blood glucose level measurements and/or indicators of blood glucose measurements obtained over a period of time. For example, the machine learning input data 206 can include a time series of blood glucose level measurements and/or indicators of blood glucose measurements obtained from a continuous blood glucose monitor. In at least some examples, the machine learning input data 206 can include blood glucose level measurements taken over a period of time, such as at least 30 minutes, at least 45 minutes, at least 60 minutes, at least 75 minutes, at least 90 minutes, or at least 120 minutes. In one or more additional examples, the machine learning input data 206 can include blood glucose level measurements captured periodically over the period of time, such as every minute, every two minutes, every three minutes, every four minutes, every five minutes, every 7 minutes, or every 10 minutes.

The machine learning input data 206 can also include carbohydrate consumption data indicating an amount of carbohydrates consumed by an individual at the intervals corresponding to the blood glucose level measurements. To illustrate, in scenarios where the blood glucose measurements are captured every four minutes, the carbohydrate consumption data can indicate an amount of carbohydrates consumed by the individual every four minutes. In one or more illustrative examples, the amount of carbohydrates consumed by an individual at a given time can be expressed in a mass of the carbohydrates consumed, such as a number of grams of carbohydrates.

Additionally, the machine learning input data 206 can include insulin-on-board measurements. In one or more examples, the machine learning input data 206 can include previous insulin-on-board levels for an individual. In one or more additional examples, the machine learning input data 206 can include one or more predicted insulin-on-board levels, such as insulin-on-board levels in at least the next 10 minutes, at least the next 15 minutes, at least the next 20 minutes, at least the next 30 minutes, at least the next 45 minutes, or at least the next 60 minutes. Insulin-on-board levels can include estimates of the amount of insulin within the blood of an individual at a given period of time. In at least some scenarios, insulin-on-board can be estimated based on a time that an individual most recently obtained an insulin dose. Further, the insulin-on-board can be estimated based on historical data indicating the reduction in insulin levels in the blood of the individual over time. Further, the machine learning input data 206 can include insulin dose data that indicates one or more times that individuals have received a dose of insulin and corresponding amounts of the insulin doses. The doses of insulin can be provided to the individuals via one or more injections.

In various examples, the machine learning training data 206 can include an amount of change in blood glucose levels and/or estimated blood glucose levels of individuals between a most recent time that a blood glucose measurement was collected and a previous time that an indicator of blood glucose measurement was collected in relation to the most recent time. For example, in scenarios where blood glucose level measurements and/or indicators of blood glucose measurements are collected every seven minutes, the machine learning training data 206 can include a change in blood glucose levels that occurs in the seven-minute intervals.

In one or more examples, the one or more convolutional neural networks 204 can include a number of convolutional layers that analyze the machine learning input data 206 to generate a first output. The first output can then be provided to one or more normalization layers. In various examples, the normalization layers can generate a second output that is provided to one or more dilation layers. In one or more illustrative examples, the one or more dilation layers can be arranged in series such that at least a portion of the output of at least a portion of the one or more dilation layers is provided to a subsequent dilation layer. In at least some examples, the output of the one or more convolutional neural networks 204 can include output from at least a portion of the dilation layers.

In one or more examples, the one or more dilation layers can correspond to one or more dilation rates. The dilation rates can be defined gaps in the data being analyzed by the one or more convolutional neural networks 204. In at least some examples, defined portions of the machine learning input data 206 are skipped with the non-skipped portions of the machine learning input data 206 being analyzed by the one or more dilation layers. In situations where the machine learning input data 206 includes at least one of indicators of blood glucose levels captured in a time series or carbohydrate consumption data indicating an amount of carbohydrates consumed over a period of time, the dilation layers can process every second data point, every third data point, every fourth data point, every fifth data point, every sixth data point, every seventh data point, every eighth data point, up to every sixteenth data point or every thirty-second data point.

In at least some examples, the one or more convolutional neural networks 204 can include one or more additional layers. To illustrate, the one or more convolutional neural networks 204 can include one or more flattening layers and/or one or more fully connected layers. In addition, the one or more convolutional neural networks 204 can include one or more layers that implement a softmax function. In various examples, the convolutional neural network can include one or more residual layers. The one or more residual layers can generate a first output that is fed back into one or more convolution layers of the one or more convolutional neural networks.

In various examples, the output of the one or more convolutional neural networks 204 can be input to a first artificial neural network 208 and to a second artificial neural network 210. The first artificial neural network 208 can determine a mean predicted value 212 for blood glucose levels of one or more individuals. In addition, the second artificial neural network 210 can determine a standard deviation 214 for the mean predicted value 212. In one or more examples, the first artificial neural network 208 can include one or more first activation functions and the second artificial neural network 210 can include one or more second activation functions. In one or more illustrative examples, the mean predicted value 212 for blood glucose levels of one or more individuals can include a change in blood glucose levels of the individual over a period of time. For example, the mean predicted value 212 for blood glucose levels of one or more individuals can include a predicted difference between the blood glucose level of the one or more individuals at a first time and the blood glucose level of the one or more individuals at a second time that is at least 2 minutes, at least 3 minutes, at least 4 minutes, at least 5 minutes, at least 6 minutes, at least 7 minutes, at least 8 minutes, at least 9 minutes, or at least 10 minutes after the first time.

The mean predicted value 212 for blood glucose levels of one or more individuals in conjunction with the standard deviation 214 can produce a predicted blood glucose distribution 216. In one or more illustrative examples, the predicted blood glucose distribution 216 can include a normal distribution of predicted blood glucose values. In various examples, at least some existing systems generate single values for blood glucose measurements and/or predict blood glucose measurements using a classification machine learning architecture. In contrast, the implementations described herein use dual artificial neural networks to separately analyze information generated by a number of convolutional layers to generate a mean predicted blood glucose value and a standard deviation that corresponds to the mean. In this way, the implementations described herein reduce the computational resources used to predict blood glucose levels of subjects and provide more accurate predictions of blood glucose levels of patients than existing systems.

In one or more examples, the machine learning architecture 202 can generate a global prediction model 218. The global prediction model 218 can be trained using population training data 220. The global prediction model 218 can include one or more parameters and one or more weights that can be tuned during the training process. In various examples, the populations training data 220 can be extracted from the machine learning input data 206. In at least some examples, the population training data 220 can be obtained from medical literature and/or longitudinal studies that provide data that corresponds to the features measured by the machine learning input data 206.

In one or more additional examples, the global prediction model 218 can be further trained to produce a customized prediction model 222. The customized prediction model 222 can be trained using individual patient training data 224. For example, as data is collected over time for a specific individual that corresponds to blood glucose levels, carbohydrate consumption, physical activity, insulin-on-board, insulin dose data, and/or insulin sensitivity, the parameters and/or weights of the global prediction model 218 can be modified to generate the customized prediction model 222. In this way, over time, the customized prediction model 222 can generate more accurate predictions for the blood glucose level of the patient. In one or more illustrative examples, at least one of the global prediction model 218 and the customized prediction model 222 can be trained using one or more stochastic gradient descent techniques.

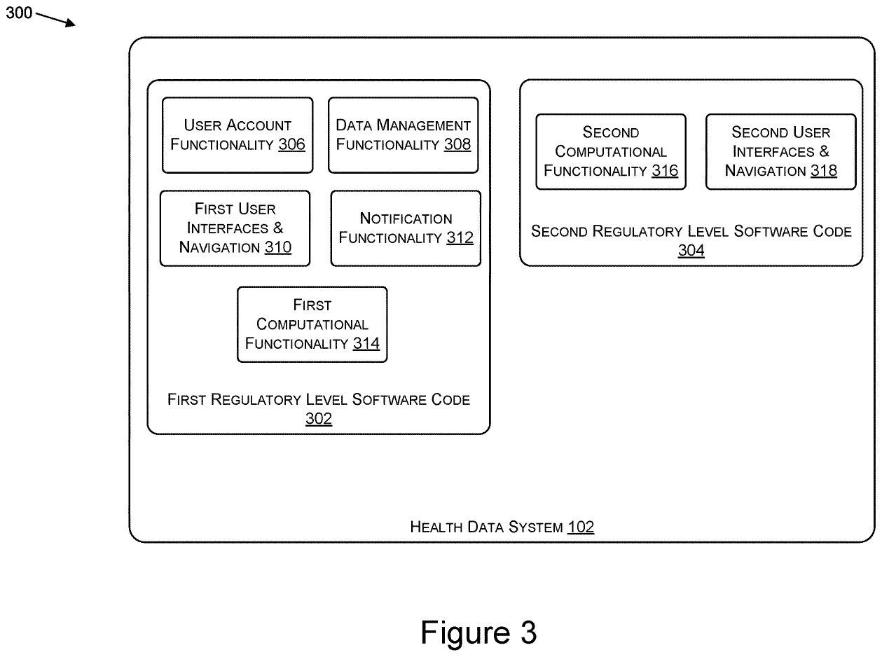

FIG. 3 illustrates an example framework 300 that stores and executes software code according to different levels of a regulatory scheme, according to one or more example implementations. The framework 300 can include first regulatory level software code 302 and second regulatory level software code 304. The first regulatory level software code 302 can correspond to operations of the health data system 102 that are regulated under a first set of regulations and the second regulatory level software code 304 can corresponds to operations of the health data system 102 that are regulated under a second set of regulations. In one or more illustrative examples, the first set of regulations and the second set of regulations can be promulgated by one or more governmental agencies. In one or more additional illustrative examples, the first set of regulations and the second set of regulations can be promulgated as part of one or more industry consortia. In one or more further illustrative examples, the first set of regulations and the second set of regulations can be promulgated by one or more industry standard setting agencies.

In various examples, the second set of regulations can be more stringent than the first set of regulations. In one or more examples, changes to the second regulatory level software code 304 can be subject to additional regulatory approval under the second set of regulations. Additionally, changes to the first regulatory level software code 302 may not be subject to additional regulatory approval under the first set of regulations. In this way, by separating the operations performed by the health data system 102 in conjunction with the first regulatory level software code 302 from the operations performed by the health data system 102 in conjunction with the second regulatory level software code 304, the framework 300 provides an efficient architecture that avoids making changes to the first regulatory level software code 302 in response to changes to the second regulatory level software code 304 and avoids unnecessary regulatory examination of the first regulatory level software code 302 that would be performed using conventional frameworks and architectures.

In one or more examples, the first regulatory level software code 302 can be stored in one or more storage locations that are separate from one or more storage locations of the second regulatory level software code 304. For example, the first regulatory level software code 302 can be stored in a first data repository included in or accessible to the health data system 102 and the second regulatory level software code 304 can be stored in a second data repository included in or accessible to the health data system 102. Additionally, the first regulatory level software code 302 can be executed separately from the second regulatory level software code 304. To illustrate, the first regulatory level software code 302 can be executed using one or more first processing units and the second regulatory level software code 304 can be executed using one or more second processing units.

In the illustrative example of FIG. 3, the first regulatory level software code 302 can include user account functionality 306. The user account functionality 306 can include operations that correspond to initiating and maintaining user accounts. For example, the user account functionality 306 can correspond to collecting and/or updating user personal information, user settings preferences, user notification preferences, preferences related to functionality of a diabetes healthcare application, one or more combinations thereof, and so forth. The first regulatory level software code 302 can also include data management functionality 308 that corresponds to operations performed by the health data system 102 to collect and store data. In at least some examples, at least a portion of the data management functionality 308 can correspond to operations performed by the data extraction and processing system 104 described in relation to FIG. 1.

Additionally, the first regulatory level software code 302 can include first user interfaces and navigation 310. The first user interfaces and navigation 310 can correspond to user interfaces of a diabetes healthcare application that collect information from users in relation to user accounts and also in relation to one or more logging functions. The one or more logging functions can include collecting physical activity data, carbohydrate consumption data, blood glucose levels, insulin-related activity, one or more combinations thereof, and the like. Further, the first regulatory level software code 302 can include notification functionality 312. The notification functionality 312 can indicate user preferences for communications channels to receive notifications and/or content of notifications. Further, the first regulatory level software code 302 can include first computational functionality 314. In various examples, the first computational functionality 314 can include operations performed by the health data system 102 in relation to the machine learning system 106 described in relation to FIG. 1 and the machine learning architecture 202 described in relation to FIG. 2.

The second regulatory level software code 304 can include second computational functionality 316 that can correspond to operations performed by the health data system 102 in relation to the blood glucose adjustment recommendation system 130. In addition, the second regulatory level software code 304 can include second user interfaces and navigation 318. The second user interfaces and navigation 318 can correspond to user interfaces and other operations performed by the health data system 102 in conjunction with communicating the blood glucose adjustment recommendation 132 described in relation to FIG. 3.

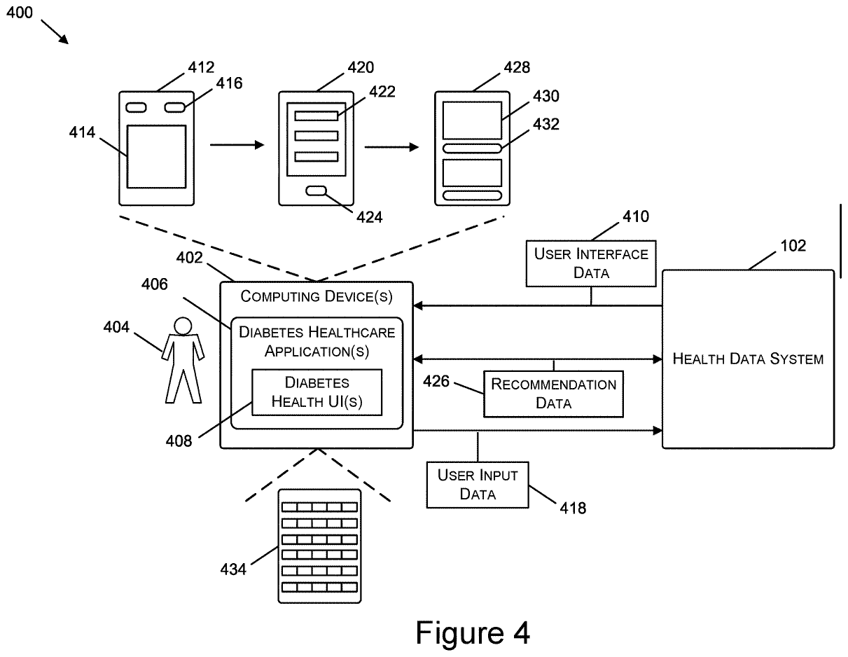

FIG. 4 illustrates an example framework 400 to capture user input and display recommendations to adjust a blood glucose level of a patient, according to one or more example implementations. The framework 400 can include the health data system 102 described with respect to FIGS. 1-3. The health data system 102 can obtain data from subjects that can be used to determine blood glucose adjustment recommendations. The blood glucose adjustment recommendations can indicate one or more actions to be performed by at least one of subjects or healthcare practitioners to adjust a blood glucose level of the subjects. In at least some examples, the blood glucose adjustment recommendations can include at least one of an amount of insulin to be administered to the subjects, an amount of carbohydrates to be consumed by the subjects, or an indication to modify physical activity.

In various examples, the carbohydrates consumed by the subjects in response to a blood glucose adjustment recommendation can include simple carbohydrates that are metabolized relatively quickly in relation to complex carbohydrates. In one or more examples, simple carbohydrates can include monosaccharides and/or disaccharides. For example, simple carbohydrates can include at least one of glucose, fructose, galactose, sucrose, or lactose. In one or more illustrative examples, the blood glucose adjustment recommendation can indicate an amount of food and/or liquid to consume to modify blood glucose level of patients or an amount of one or more simple carbohydrates to consume to modify the blood glucose levels of patients. In one or more additional examples, complex carbohydrates can include at least one of oligosaccharides or polysaccharides.

In addition, the framework 400 can include one or more computing devices 402 operated by a subject 404. The one or more computing devices 402 can include a laptop computing device, a tablet computing device, a mobile computing device, a smartphone, a smartwatch, a virtual reality headset, an augmented reality headset, smart glasses, a medical device, one or more combinations thereof, and the like. The subject 404 can include a subject in which a diabetic biological condition is present or a healthcare practitioner treating a subject in which a diabetic biological condition is present.

The one or more computing devices 402 can execute one or more diabetes healthcare applications 406. The one or more diabetes healthcare applications 406 can include the diabetes healthcare application 112 described with respect to FIG. 1. The one or more diabetes healthcare applications 406 can obtain data that is sent to the health data system 102. In one or more examples, the one or more diabetes healthcare applications 406 can determine blood glucose adjustment recommendations based on data obtained by the one or more diabetes healthcare applications 406. In one or more additional examples, data obtained by the one or more diabetes healthcare applications 406 can be used by the health data system 102 to determine blood glucose adjustment recommendations. The one or more diabetes healthcare applications 406 can also display blood glucose adjustment recommendations. In at least some examples, the one or more diabetes healthcare applications 406 can include a first diabetes healthcare application and a second diabetes healthcare application that is integrated with the first diabetes healthcare application. In these scenarios, at least a portion of the functionality of the second diabetes healthcare application can be accessed via the first diabetes healthcare application. In at least some examples, at least a portion of the functionality of the second diabetes healthcare application can be integrated with the first diabetes healthcare application using one or more application programming interface calls of at least one of the first diabetes healthcare application or the second diabetes healthcare application.

In one or more illustrative examples, the first diabetes healthcare application can be operated in conjunction with one or more medical devices and/or sensors that can be used to monitor blood glucose levels of subjects and the second diabetes healthcare application can be used to calculate blood glucose adjustment recommends and/or to predict future blood glucose levels of subjects. In one or more additional illustrative examples, the first diabetes healthcare application can be used to at least one of collect or monitor diabetes related information about subjects and the second diabetes healthcare application can be used to calculate blood glucose adjustment recommendations and/or to predict future blood glucose levels of subjects. In various examples, the second diabetes healthcare application can also be used to obtain at least a portion of the data used to determine blood glucose adjustment recommendations. In one or more examples, the first diabetes healthcare application can be at least one of controlled, maintained, or administered by one or more first entities and the second diabetes healthcare application can be at least one of controlled, maintained, or administered by one or more second entities.

The one or more diabetes healthcare applications 406 can be executed to display one or more user diabetes health user interfaces 408. The diabetes health user interfaces 408 can be used to capture data from subjects that can be used to determine blood glucose modification recommendations. In one or more additional examples, the one or more diabetes health user interfaces 408 can be used to capture data from subjects that can be used to forecast future blood glucose levels of subjects. In at least some examples, the health data system 102 can send user interface data 410 to the one or more computing devices 402. The one or more diabetes healthcare applications 406 can use the user interface data 410 to generate the one or more diabetes health user interfaces 408. In various examples, the user interface data 410 can include information corresponding to at least one of layouts or appearances of user interface elements included in the one or more diabetes health user interfaces 408. Additionally, the user interface data 410 can include information corresponding to functionality related to one or more user interface elements included in the one or more diabetes health user interfaces 408.

The one or more diabetes health user interfaces 408 can include one or more data capture user interfaces, such as an example data capture user interface 412. The example data capture user interface 412 can include one or more first information displaying user interface elements 414 and one or more first input capture user interface elements 416. The one or more first information displaying user interface elements 414 can include at least one of text content, image content, or video content related to diabetes health. Additionally, the one or more first input capture user interface elements 416 can be selectable to obtain input from users of the one or more diabetes healthcare applications 406. In one or more illustrative examples, the one or more first input capture user interface elements 416 can include at least one of one or more drop down menus, one or more text fields, one or more file upload sections, one or more radio buttons, one or more date and/or time selection fields, one or more stepper buttons, one or more list boxes, one or more sliders, one or more combinations thereof, and so forth.

In one or more illustrative examples, the one or more data capture user interfaces 412 can include one or more insulin dose user interfaces. The one or more insulin dose user interfaces can be used to capture one or more amounts of insulin administered to the subject 404 and times that the one or more insulin doses were administered. In various examples, the one or more insulin dose user interfaces can indicate one or more previous doses of insulin administered to the subject 404. In one or more additional examples, the one or more insulin dose user interfaces can include one or more user interface elements to confirm one or more insulin doses administered to a subject and/or one or more user interface elements related to provide reminders regarding administering insulin doses.

In one or more additional illustrative examples, the one or more data capture user interfaces 412 can include one or more glucose level user interfaces. The one or more glucose level user interfaces can include one or more user interface elements to capture a blood glucose level of subjects. The blood glucose level captured by the one or more glucose level user interfaces can correspond to a current blood glucose level or a recent blood glucose level. In various examples, the blood glucose level captured by the glucose level user interfaces can correspond to a blood glucose level of subjects within a threshold amount of time from a current time. In at least some examples, the blood glucose level captured by the one or more glucose level user interfaces can be a blood glucose level determined by one or more blood glucose level monitoring devices within no greater than 30 minutes with respect to a current time, within no greater than 20 minutes with respect to a current time, within no greater than 15 minutes with respect to a current time, within no greater than 10 minutes with respect to a current time, within no greater than 5 minutes of a current time, or within no greater than 2 minutes with respect to a current time. In one or more examples, a current blood glucose level of a subject can include a blood glucose level determined by one or more blood glucose level monitoring devices within no greater than 30 seconds with respect to a current time, within no greater than 60 seconds with respect to a current time, within no greater than 2 minutes with respect to a current time, within no greater than 5 minutes with respect to a current time, or within no greater than 10 minutes with respect to a current time.

In one or more additional examples, the blood glucose level of a subject can be received from at least one of a blood glucose level monitoring device or a diabetes healthcare application 406 in electronic communication with a blood glucose level monitoring device. In still other examples, the blood glucose level of a subject can be obtained from the health data system 102. For example, the health data system 102 can be in electronic communication with a blood glucose level monitoring device and/or with a computing device in communication with a blood glucose level monitoring device to record blood glucose levels of the subject at one or more intervals. Further, the blood glucose level of a subject can be manually entered by the subject via one or more user interface elements of the one or more glucose level user interfaces. In one or more scenarios, the one or more glucose level user interfaces can include one or more user interface elements that are selectable to request a current blood glucose level from at least one of a diabetes healthcare application 406, the health data system 102, or a blood glucose level monitoring device.

In at least some examples, the one or more glucose level user interfaces can obtain user input confirming a current blood glucose level or a recent blood glucose level of a subject. In various examples, in situations where a most recent blood glucose level of a patient was obtained more than a threshold amount of time from a current time, the one or more glucose level user interfaces can show a request to enter a more recently obtained blood glucose level for the subject. To illustrate, the one or more glucose level user interfaces can indicate that a blood glucose modification recommendation will not be calculated unless a blood glucose level of a subject is obtained within a threshold period of time from a current time. In these implementations, the threshold period of time can be within no greater than 30 minutes with respect to a current time, within no greater than 20 minutes with respect to a current time, within no greater than 15 minutes with respect to a current time, within no greater than 10 minutes with respect to a current time, within no greater than 5 minutes of a current time, or within no greater than 2 minutes with respect to a current time.

In one or more further illustrative examples, the one or more data capture user interfaces 412 can include one or more carbohydrate consumption user interfaces. The one or more carbohydrate consumption user interfaces can be used to capture amounts of carbohydrates consumed by subjects over a period of time. In various examples, the one or more carbohydrate consumption user interfaces can include one or more user interface elements to capture amounts of carbohydrates consumed by subjects within an additional threshold period of time. In one or more implementations, the additional threshold period of time can include within no greater than 2 hours with respect to a current time, within no greater than 90 minutes with respect to a current time, within no greater than 60 minutes with respect to a current time, within no greater than 45 minutes with respect to a current time, within no greater than 30 minutes with respect to a current time, within no greater than 20 minutes with respect to a current time, within no greater than 15 minutes with respect to a current time, or within no greater than 10 minutes with respect to a current time. In various examples, the one or more carbohydrate consumption user interfaces can include one or more user interface elements to capture amounts of simple carbohydrates consumed by subjects within the additional threshold period of time. In still other examples, the one or more carbohydrate consumption user interfaces can include one or more user interface elements to capture a first amount of simple carbohydrates and a second amount of complex carbohydrates consumed by subjects within one or more additional threshold periods of time. In various examples, the one or more carbohydrate consumption user interfaces can include one or more user interface elements to capture an amount of simple carbohydrates consumed within a first threshold period of time and an amount of complex carbohydrates consumed within a second threshold period of time that is greater than the first threshold period of time. In at least some examples, the first threshold period of time can be less than the second threshold period of time. To illustrate, the first threshold period of time can be no greater than 30 minutes, no greater than 20 minutes, no greater than 15 minutes, no greater than 10 minutes, no greater than 5 minutes, or no greater than 2 minutes from a current time and the second threshold period of time can be no greater than 120 minutes, no greater than 90 minutes, no greater than 60 minutes, no greater than 45 minutes, no greater than 30 minutes, no greater than 20 minutes, or no greater than 15 minutes from a current time.

In still other illustrative examples, the one or more data capture user interfaces 412 can include one or more physical activity user interfaces. The one or more physical activity user interfaces can include one or more user interface elements to capture an amount of physical activity performed by subjects within a threshold period of time. In one or more examples, the threshold period of time can correspond to a period of time between a current time and a time that subjects began physical activity. In one or more additional examples, the threshold period of time can correspond to a period of time between a current time and a time that subjects completed physical activity. In various examples, the threshold period of time can include no greater than 6 hours, no greater than 5 hours, no greater than 4 hours, no greater than 3 hours, no greater than 120 minutes, no greater than 90 minutes, no greater than 60 minutes, no greater than 45 minutes, no greater than 30 minutes, no greater than 15 minutes, no greater than 10 minutes, or no greater than 5 minutes with respect to a current time. Additionally, the one or more physical activity user interface elements can include one or more user interface elements to capture an amount of time for which the physical activity was performed. Further, the one or more physical activity user interface elements can include one or more user interface elements to capture an intensity of the physical activity. The intensity of the physical activity can correspond to a type of physical activity performed by subjects and/or a heart rate achieved by the subjects during the physical activity. In at least some examples, the one or more physical activity user interfaces can include one or more user interface elements to capture a duration of physical activity performed by subjects. In one or more further implementations, the one or more physical activity user interfaces can capture data indicating heart rate information such as a current heart rate of subjects, an average resting heart rate of the subjects, a difference between current heart rate and average resting heart rate, combinations thereof, and the like. In various scenarios, the heart rate information can also be provided to the one or more diabetes healthcare applications 406 from one or more computing devices, one or more wearable devices, and/or one or more sensors that gather personal health data from subjects.

In various examples, information obtained by the one or more data capture user interfaces 412 can be used to generate user input data 418 that is sent to the health data system 102. The user input data 418 can include at least one of insulin dose information, blood glucose level information, carbohydrate consumption information, physical activity information, one or more combinations thereof, and the like.

In one or more examples, the one or more data capture user interfaces 412 displayed by the diabetes health application can include a sequence of user interfaces with input being obtained from subjects before moving to a next user interface in the sequence. For example, the one or more data capture user interfaces can include a first user interface to capture information about one or more previous insulin doses administered to the subject 404. In response to receiving input related to the one or more previous doses of insulin administered to the subject 404, a second data capture user interface can be displayed to obtain information related to blood glucose levels of the subject 404. In response to receiving the information related to the blood glucose levels of the subject 404, a third data capture user interface can be displayed to obtain information related to an amount of carbohydrates consumed by the subject 404. In response to capturing input in relation to the amount of carbohydrates consumed by the subject 404, a fourth data capture user interface can be displayed to obtain information related to physical activity performed by the subject 404. In at least some examples, after a blood glucose modification recommendation has been given via a user interface in the sequence of user interfaces, the information used for the blood glucose modification recommendation cannot be changed unless the sequence of user interfaces is restarted in relation to a new blood glucose modification recommendation. In various examples, the completion of capturing information from each of the data capture user interfaces 412 takes place before calculations are performed to generate blood glucose modification recommendations. In still other examples, one or more portions of the user input data 418 can be used to perform calculations in relation to blood glucose modification recommendations in response to information being captured by one or more of the data capture user interfaces 412.

The diabetes health user interfaces 408 can also include one or more information review user interfaces 420. The one or more information review user interfaces 420 can include one or more user second information displaying user interface elements 422 and one or more second input capture user interface elements 424. In various examples, the one or more second information displaying user interface elements 422 can display information captured via the one or more data capture user interfaces 412. In at least some examples, the one or more second input capture user interface elements 424 can be selectable to cause the health data system 102 to determine one or more blood glucose modification recommendations. In one or more additional examples, the one or more second data capture user interface elements 424 can be selectable to cause the user input data 418 to be used to perform calculations to determine blood glucose modification recommendations.

In one or more illustrative examples, the one or more information review user interfaces 420 can be displayed after a sequence of a number of data capture user interfaces 412 has completed. In various examples, by causing the one or more review user interfaces 420 to be displayed after completion of a sequence of the one or more information review user interfaces 420, the information used to generate blood glucose modification recommendations by the health data system 102 can be controlled and can result in more accurate blood glucose modification recommendations in relation to scenarios where subjects can obtain blood glucose modification recommendations with incomplete and/or inaccurate information. Further, after generating the one or more information review user interfaces 420 for a given session to calculate a blood glucose modification recommendation and after obtaining input confirming the information entered via the one or more data capture user interfaces 412, information entered via one or more of the data capture user interfaces 412 is unable to be modified. In at least some examples, information entered via each of the data capture user interfaces 412 is unable to be modified in response to generating and displaying the one or more information review user interfaces 420. In this way, individual sessions for determining blood glucose modification recommendations are based on a single set of inputs obtained from individual data capture user interfaces 412 to provide data integrity, increase the accuracy of blood glucose modification recommendations, and to prevent users from performing actions related to blood glucose modification based on an incomplete and/or inaccurate set of information. In situations where the subject 404 wants to change the data used to calculate a blood glucose modification recommendation, a new session can be initiated by the subject 404 and the series of one or more data capture user interfaces 412 can be restarted.

Based on the user input data 418, at least one of the one or more diabetes healthcare applications 406 or the health data system 102 can generate one or more blood glucose modification recommendations. The blood glucose modification recommendations can be generated according to one or more implementations described with respect to FIGS. 1, 2, 3, 5, and/or 6. The blood glucose modification recommendations can be stored by one or more data storage devices included in and/or in electronic communication with the health data system 102. In one or more examples, the blood glucose modification recommendations generated with respect to the subject 404 can be stored in association with an account of the subject 404. The account of the subject 404 can be related to at least one of the health data system 102 or the one or more diabetes healthcare applications 406. In various examples, the one or more diabetes healthcare applications 406 can send the recommendation data 426 to the health data system 102 after generating one or more blood glucose modification recommendations for the subject 404. In at least some examples, at least a portion of the computations to generate blood glucose modification recommendations can be performed by the one or more diabetes healthcare applications 406 being executed by the one or more computing devices 402 in accordance with one or more regulatory schemes. That is, in one or more scenarios, software code to perform computations to generate blood glucose modification recommendations can be stored in memory of the one or more computing devices and executed in conjunction with the one or more diabetes healthcare applications 406 to comply with one or more regulatory schemes. Further, in situations where blood glucose modification recommendation computations are performed by the one or more diabetes healthcare applications 406, blood glucose modification recommendations can be generated in an offline mode where connectivity to one or more communications networks is unavailable at a given time. In this way, safety for subjects can increase in that subjects are able to obtain blood glucose modification recommendations even in scenarios where network communication may be unavailable.

The blood glucose modification recommendations can be included in recommendation data 426 generated by at least one of the one or more diabetes healthcare applications 406 or the health data system 102. The recommendation data 426 can include information related to blood glucose modification recommendation. For example, the recommendation data 426 can indicate an amount of insulin to be administered to the subject 404, an amount of carbohydrates to be consumed by the subject, an indication to refrain from physical activity for a period of time, an indication to alter an intensity of physical activity, an indication to contact a healthcare practitioner, an indication regarding blood glucose levels of the subject 404 being outside of a desired range of blood glucose levels, or one or more combinations thereof.

In various examples, the recommendation data 426 can be used to generate one or more recommendation user interfaces 428. The one or more recommendation user interfaces 428 can be displayed via the one or more diabetes healthcare applications 406. At least a portion of the data used to generate the one or more recommendation user interfaces 428 can be included in the user interface data 410. The one or more recommendation user interfaces 428 can include one or more third information displaying user interface elements 430 and one or more third input capture user interface elements 432. The one or more third information displaying user interface elements 430 can include information included in the recommendation data 426. In one or more illustrative examples, the one or more third information displaying user interface elements can include a blood glucose modification recommendation.

In one or more examples, the one or more third input capture user interface elements 432 can obtain input indicating one or more actions performed by the subject 404 in relation to the blood glucose modification recommendation. For example, the one or more third input capture user interface elements 432 can obtain information indicating an actual insulin dose administered to the subject 404 and/or an actual amount of carbohydrates consumed by the subject 404. In one or more additional examples, the one or more third information displaying elements 430 can indicate differences between the blood glucose recommendation provided to the subject 404 via the one or more diabetes healthcare applications 406 and an action taken in relation to the subject 404 in response to the blood glucose modification recommendation. In one or more illustrative examples, a blood glucose modification recommendation can be displayed in accordance with one or more display features, such as font size, text color, text size, and the like, that are different from those of the actual action performed in relation to the subject 404 in situations where the action performed in relation to the subject 404 differs from the blood glucose modification recommendation. In this way, changes made to the blood glucose modification recommendation by at least one of the subject 404 or a healthcare practitioner can be highlighted in the one or more recommendation user interfaces 428. The one or more third input capture user interface elements 432 can also include one or more user interface elements that are selectable to save the blood glucose modification recommendation in relation to the subject 404 or to delete the blood glucose modification recommendation.

The one or more diabetes healthcare applications 406 can also be executable to display one or more logbook user interfaces 434. The one or more logbook user interfaces 434 can capture information and/or display information related to treatment of one or more diabetes-related biological conditions with respect to subjects. In one or more examples, the one or more logbook user interfaces 434 can capture input indicating insulin doses administered to subjects, glucose levels of subjects, physical activity performed by subjects, carbohydrates consumed by subjects, one or more other actions performed in relation to treatment of one or more diabetes-related biological conditions, or one or more combinations thereof. In one or more additional examples, the one or more logbook user interfaces 434 can capture input indicating timing of one or more activities performed in relation to treatment of one or more diabetes-related biological conditions.

In one or more illustrative examples, the one or more logbook user interfaces 434 can indicate a time in which one or more activities were performed and an amount related to the one or more activities. For example, the one or more logbook user interfaces 434 can indicate one or more times and one or more amounts of one or more insulin doses administered to the subject 404. In addition, the one or more logbook user interfaces 434 can indicate one or more times and one or more amounts of carbohydrates consumed by the subject 404. In at least some examples, the one or more logbook user interfaces 434 can indicate types of carbohydrates consumed by the subject 404. Further, the one or more logbook user interfaces 434 can indicate one or more times and one or more amounts of physical activity performed by the subject 404. In various examples, the one or more logbook user interfaces 434 can indicate a type and/or an intensity of physical activity performed by the subject 404. In still other examples, the one or more logbook user interfaces 434 can indicate glucose levels of the subject 404 at one or more times. In one or more scenarios, the glucose levels of the subject 404 displayed in the one or more logbook user interfaces 434 can be obtained via input from the subject 404 and/or via one or more glucose level monitoring devices. In one or more additional implementations, the one or more logbook user interfaces 434 can indicate changes made by the subject 404 in relation to blood glucose modification recommendations provided by the health data system 102. In one or more further implementations, modifications to logbook entries included in the one or more logbook user interfaces 434 can be restricted. To illustrate, the one or more diabetes healthcare applications 406 can restrict modifications to logbook entries to at least one of amounts of carbohydrates consume by the subject 404 or doses of insulin administered to the subject 404. In one or more examples, information included in the one or more logbook user interfaces 434 can be used to generate at least one of the one or more data capture user interfaces 412 or the one or more information review user interfaces 420.

Although the illustrative one or more diabetes health user interfaces 408 have been described as being displayed in conjunction with the one or more diabetes healthcare applications 406, in one or more additional implementations, at least a portion of the one or more diabetes health user interfaces 408 can be displayed using a browser application. Additionally, in one or more illustrative examples, at least a portion of the user interfaces 412, 420, 428 can be implemented with respect to the first user interfaces and navigation 310 as described in relation to FIG. 3. In still other examples, at least a portion of the user interfaces 412, 420, 428 can be implemented with respect to the second user interfaces and navigation 318 as described in relation to FIG. 3.

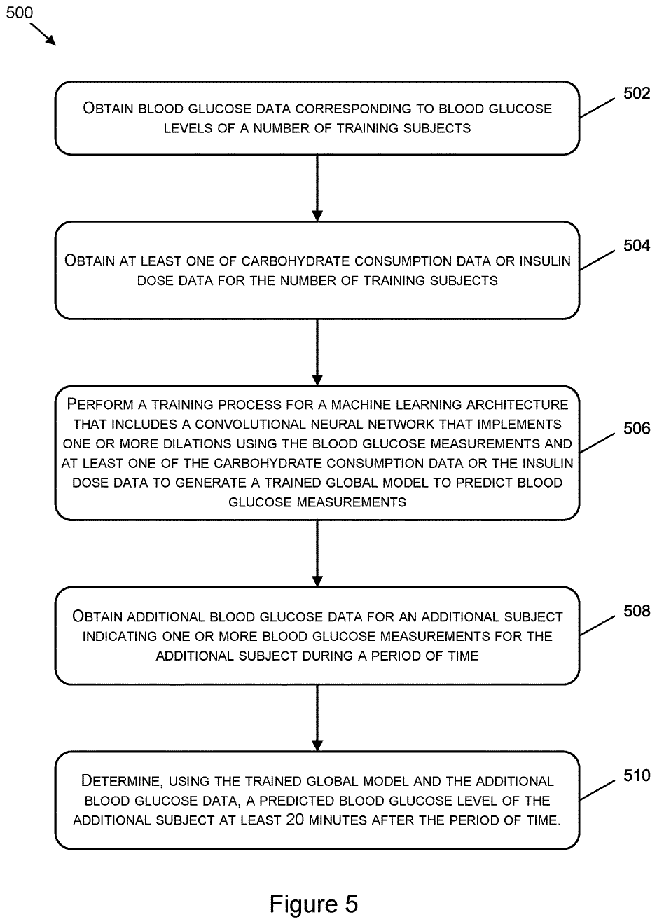

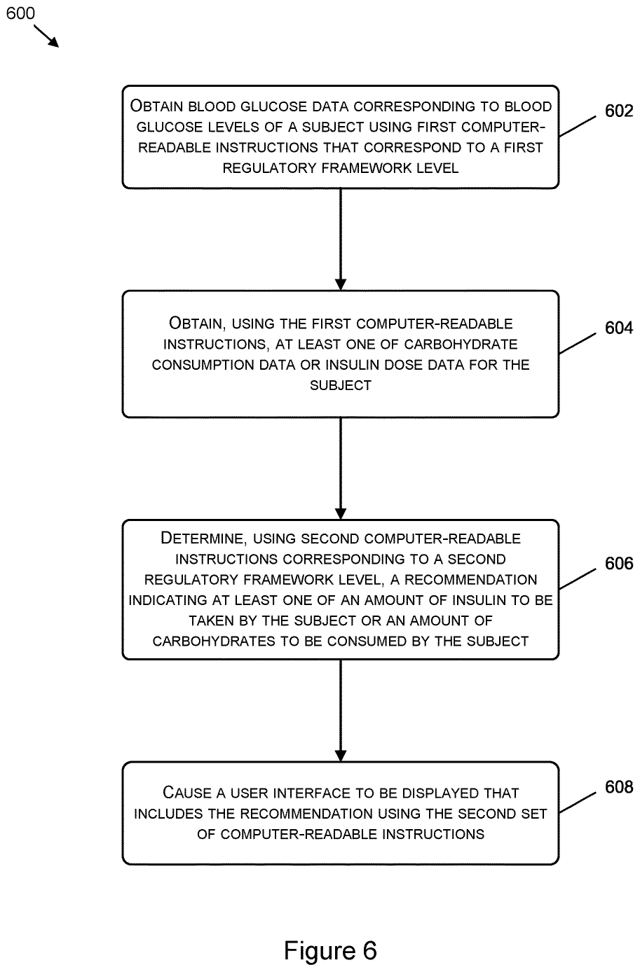

FIGS. 5 and 6 illustrate example methods for predicting blood glucose levels of individuals and for generating treatment recommendations for individuals in which diabetes is present. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

FIG. 5 is a flow diagram of an example process 500 to predict a blood glucose level of a patient using a machine learning architecture, according to one or more example implementations. The process 500 can include, at 502, obtaining blood glucose data corresponding to blood glucose levels of a number of training subjects. At 504, the process 500 can include obtaining at least one of carbohydrate consumption data or insulin dose data for the number of training subjects. The carbohydrate consumption data can indicate an amount of carbohydrates consumed by at least a portion of the number of training subjects with respect to at least a portion of the blood glucose levels of the at least a portion of the number of training subjects. Additionally, the insulin dose data can indicate one or more times that at least a portion of the number of training subjects received an amount of insulin and a dose of the amount of insulin.

The process 500 can also include, at 506, performing a training process for a machine learning architecture that includes a convolutional neural network that implements one or more dilations using the indicators of blood glucose measurements and at least one of the carbohydrate consumption data or the insulin dose data to generate a trained global model to predict blood glucose measurements. In one or more examples, the convolutional neural network can generate intermediate data. The intermediate data can be provided to a first artificial neural network and a second artificial neural network of the machine learning architecture. The first artificial neural network can generate predicted mean value for the blood glucose level of the additional subject based on a plurality of predicted values of the blood glucose level of the additional subject generated by the first artificial neural network. Additionally, the second artificial neural network can generate a standard deviation that corresponds to the predicted mean value.

Additionally, at 508, the process 500 can include obtaining additional blood glucose data for an additional subject. The additional blood glucose data can indicate one or more blood glucose measurements for the additional subject during a period of time. Further, the process 500 can include, at 510, determining, using the trained global model and the additional blood glucose data, a predicted blood glucose level of the additional subject at least 20 minutes after the period of time. In one or more additional examples, an additional training process can be performed for the machine learning model using the trained global model and the additional blood glucose data to generate a personalized model for the additional subject. The additional training process to generate the personalized model can also use additional carbohydrate consumption data and/or additional insulin dose data for the additional subject. The additional carbohydrate consumption data can indicate an amount of carbohydrate consumption that corresponds to a number of blood glucose levels for the additional subject. The additional insulin dose data can indicate a time that the additional subject received a dose of insulin and an amount of the dose of insulin. In at least some examples, the dose of insulin can be injected into the additional subject.

In various examples, the trained global model includes a first number of components and a first number of weights that correspond to the first number of components. In at least some examples, the first number of components can correspond to parameters of the trained global model. Additionally, the personalized model can include a second number of components and a second number of weights. In at least some examples, the first number of components and the second number of components can be the same, while the values of the second number of weights and the first number of weights can be different. In this way, the personalized model can be used to predict blood glucose levels of the subject more accurately. In at least some additional examples, the values of the second number of weights can be similar to or the same as the values of the first number of weights.

FIG. 6 is a flow diagram of an example process 600 to determine a recommendation to adjust a blood glucose level of a patient using software code that is maintained and executed in accordance with different levels of a regulatory scheme, according to one or more example implementations. At 602, the process 600 includes obtaining blood glucose data corresponding to blood glucose levels of a subject using first computer-readable instructions that correspond to a first regulatory framework level. The process 600 can also include, at 604, obtaining, using the first computer-readable instructions, at least one of carbohydrate consumption data or insulin dose data for the subject. The carbohydrate consumption data can indicate an amount of carbohydrates consumed by the subject with respect to at least a portion of the blood glucose levels. The insulin dose data can indicate one or more times that the subject received an insulin dose and an amount of the dose. In at least some examples, the dose of insulin can be injected into the subject.

In various examples, the first computer-readable instructions can be executed to analyze, using a machine learning architecture, the blood glucose data to determine a predicted blood glucose level of the subject at least 20 minutes after a period of time based on at least a portion of the blood glucose levels included in the blood glucose data.

In addition, at 606, the process 600 can include determining, using second computer-readable instructions, a recommendation indicating at least one of an amount of insulin to be taken by the subject or an amount of carbohydrates to be consumed by the subject. The second computer-readable instructions can correspond to a second regulatory framework level that is different from the first regulatory framework level. In at least some examples, the recommendation is determined can be based on the predicted blood glucose level generated by the machine learning architecture. The recommendation can also be produced using physical activity data and/or insulin consumption data for the subject. The physical activity data can indicate an amount of activity performed by the subject during a period of time that corresponds to at least a portion of the blood glucose levels, wherein the physical activity data is obtained using the first set of computer-readable instructions. In addition, the insulin consumption data can indicate an amount of insulin taken by the individual during the period of time, wherein the insulin consumption data is obtained using the first set of computer-readable instructions. In one or more examples, the second regulatory framework level can include a greater number of regulations than the first regulatory framework level. In various examples, the recommendation can be generated using a carbohydrate to insulin ratio that corresponds to an amount of carbohydrates consumed by the subject over a period of time in relation to an amount of insulin received by the subject over the period of time. The carbohydrate to insulin ratio can be determined using the first set of computer-readable instructions. Additionally, the insulin sensitivity data can indicate an amount of change in blood glucose levels of the subject in response to one or more amounts of insulin and can be obtained using the first set of computer-readable instructions.

Further, the process 600 can include, at 608, causing a user interface to be displayed that includes the recommendation using the second set of computer-readable instructions. In various examples, the user interface can also indicate an amount of time that blood glucose levels of the subject have been within a target range of blood glucose levels. In one or more additional examples, one or more additional user interfaces can be generated to capture at least one of the carbohydrate consumption data, the physical activity data, or the insulin consumption data. The one or more additional user interfaces can be generated using the first set of computer-readable instructions. In one or more illustrative examples, the one or more additional user interfaces can be generated within an application executed by a computing device of the subject.

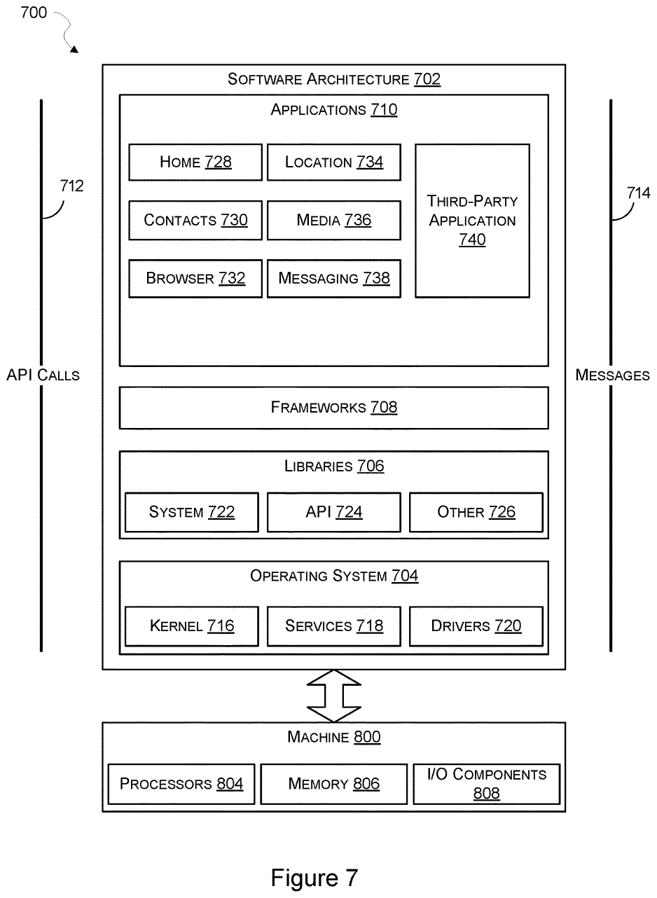

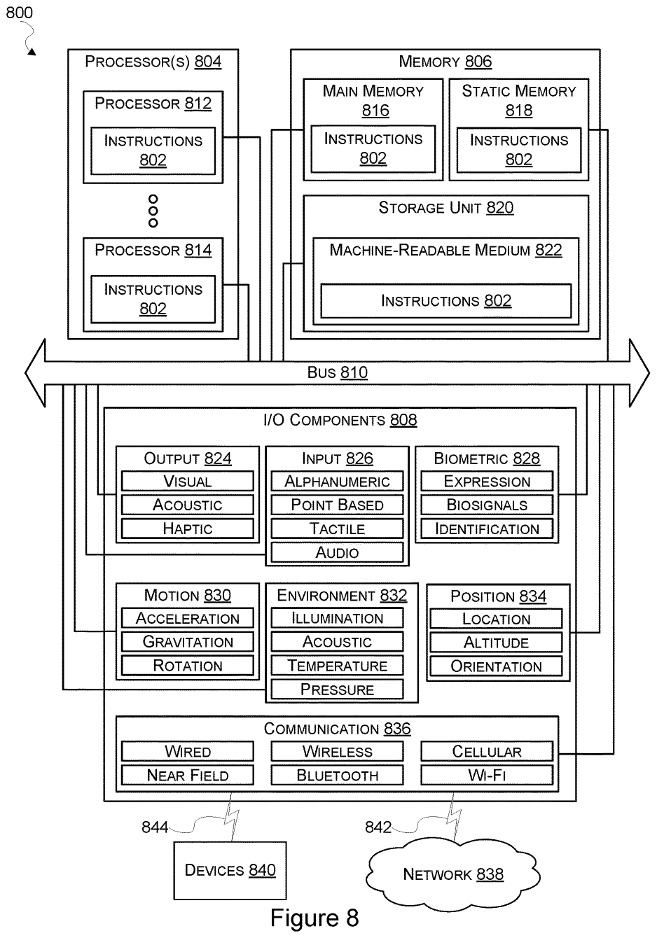

FIG. 7 is a block diagram 700 illustrating an architecture for software 702, which can be installed on any one or more of the devices described herein. FIG. 7 is merely a non-limiting example of a software architecture, and it will be appreciated that many other architectures can be implemented to facilitate the functionality described herein. In various embodiments, the software 702 can be implemented by hardware such as a machine 800 described in more detail with respect to FIG. 8 that includes processors 804, memory 806, and input/output (I/O) components 808. In this example architecture, the software 702 can be conceptualized as a stack of layers where each layer may provide a particular functionality. For example, the software 702 can include layers such as an operating system 704, libraries 706, frameworks 708, and applications 710. Operationally, the applications 710 invoke API calls 712 through the software stack and receive messages 714 in response to the API calls 712, consistent with some implementations.

In various implementations, the operating system 704 manages hardware resources and provides common services. The operating system 704 can include, for example, a kernel 716, services 718, and drivers 720. The kernel 716 can act as an abstraction layer between the hardware and the other software layers, consistent with some embodiments. For example, the kernel 716 can provide memory management, processor management (e.g., scheduling), component management, networking, and security settings, among other functionalities. The services 718 can provide other common services for the other software layers. The drivers 720 can be responsible for controlling or interfacing with the underlying hardware, according to some embodiments. For instance, the drivers 720 can include display drivers, camera drivers, BLUETOOTH® or BLUETOOTH® Low-Energy drivers, flash memory drivers, serial communication drivers (e.g., Universal Serial Bus (USB) drivers), Wi-Fi® drivers, audio drivers, power management drivers, and so forth.

In some embodiments, the libraries 706 can provide a low-level common infrastructure utilized by the applications 710. The libraries 706 can include system libraries 722 (e.g., C standard library) that can provide functions such as memory allocation functions, string manipulation functions, mathematic functions, and the like. In addition, the libraries 706 can include API libraries 724 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in 2D and 3D in a graphic context on a display), database libraries (e.g., SQLite to provide various relational database functions), web libraries (e.g., WebKit to provide web browsing functionality), and the like. The libraries 706 can also include a wide variety of other libraries 726 to provide many other APIs to the applications 710.

The frameworks 708 can provide a high-level common infrastructure that can be utilized by the applications 710, according to some implementations. For example, the frameworks 708 can provide various graphical user interface (GUI) functions, high-level resource management, high-level location services, and so forth. The frameworks 708 can provide a broad spectrum of other APIs that can be utilized by the applications 710, some of which may be specific to a particular operating system 704 or platform.

In an example implementation, the applications 710 can include a home application 728, a contacts application 730, a browser application 732, a location application 734, a media application 736, a messaging application 738, and a broad assortment of other applications, such as a third-party application 740. According to some implementations, the applications 710 can be programs that execute functions defined in the programs. Various programming languages can be employed to create one or more of the applications 710, structured in a variety of manners, such as object-oriented programming languages (e.g., Objective-C, Java, or C++) or procedural programming languages (e.g., C or assembly language). In a specific example, the third-party application 740 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 740 can invoke the API calls 712 provided by the operating system 704 to facilitate functionality described herein.

FIG. 8 illustrates a diagrammatic representation of a machine 800 in the form of a computer system within which a set of instructions 802 may be executed for causing the machine 800 to perform any one or more of the methodologies discussed herein, according to one or more example implementations. Specifically, FIG. 8 shows a diagrammatic representation of the machine 800 in the example form of a computer system, within which instructions (e.g., software, a program, an application, an applet, an app, or other executable code) for causing the machine 800 to perform any one or more of the methodologies discussed herein may be executed. For example, the instructions 802 can cause the machine 800 to execute the processes and/or operations described with respect to FIG. 1 to FIG. 5. The instructions 802 transform the general, non-programmed machine 800 into a particular machine 800 programmed to carry out the described and illustrated functions in the manner described. In alternative implementations, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 can comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 802, sequentially or otherwise, that specify actions to be taken by the machine 800. Further, while only a single machine 800 is illustrated, the term “machine” shall also be taken to include a collection of machines 800 that individually or jointly execute the instructions 802 to perform any one or more of the methodologies discussed herein.

The machine 800 can include processors 804, memory 806, and I/O components 808, which may be configured to communicate with each other such as via a bus 810. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) can include, for example, a processor 812 and a processor 814 that can execute the instructions 802. The term “processor” is intended to include multi-core processors that may comprise two or more independent processors (sometimes referred to as “cores”) that may execute instructions 802 contemporaneously. Although FIG. 8 shows multiple processors 804, the machine 800 may include a single processor 812 with a single core, a single processor 812 with multiple cores (e.g., a multi-core processor 812), multiple processors 812, 814 with a single core, multiple processors 812, 814 with multiple cores, or any combination thereof.

The memory 806 can include a main memory 816, a static memory 818, and a storage unit 820, each accessible to the processors 804 such as via the bus 810. The storage unit 820 can include at least one machine-readable medium 822. The main memory 816, the static memory 818, the storage unit 820, and/or the machine-readable medium 822 can store the instructions 802 embodying any one or more of the methodologies or functions described herein. The instructions 802 can also reside, completely or partially, within the main memory 816, within the static memory 818 within the storage unit 820, within the machine-readable medium 822, within at least one of the processors 804 (e.g., within the processor's cache memory), or any suitable combination thereof, during execution thereof by the machine 800.

The I/O components 808 can include a wide variety of components to receive input, provide output, produce output, transmit information, exchange information, capture measurements, and so on. The specific I/O components 808 that are included in a particular machine 800 can depend on the type of machine. For example, portable machines such as mobile phones can include a touch input device or other such input mechanisms, while a headless server machine will likely not include such a touch input device. It will be appreciated that the I/O components 808 can include many other components that are not shown in FIG. 8. The I/O components 808 can be grouped according to functionality merely for simplifying the following discussion, and the grouping is in no way limiting. In various example implementations, the I/O components 808 can include output components 824 and input components 826. The output components 824 can include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 826 can include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example implementations, the I/O components 808 can include biometric components 828, motion components 830, environmental components 832, and/or position components 834, among a wide array of other components. For example, the biometric components 828 can include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 830 can include acceleration sensor components (e.g., accelerometer), gravitation sensor components, rotation sensor components (e.g., gyroscope), and so forth. The environmental components 832 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that can provide indications, measurements, or signals corresponding to a surrounding physical environment. The position components 742 can include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication can be implemented using a wide variety of technologies. The I/O components 808 can include communication components 836 operable to couple the machine 800 to a network 838 or devices 840 via a coupling 842 and a coupling 844, respectively. For example, the communication components 836 can include a network interface component or another suitable device to interface with the network 838. In further examples, the communication components 836 can include wired communication components, wireless communication components, cellular communication components, near field communication (NFC) components, Bluetooth® components (e.g., Bluetooth® Low Energy), Wi-Fi® components, and other communication components to provide communication via other modalities. The devices 840 can be another machine or any of a wide variety of peripheral devices (e.g., coupled via a USB).

Moreover, the communication components 836 can detect identifiers or include components operable to detect identifiers. For example, the communication components 836 can include radio-frequency identification (RFID) tag reader components, NFC smart tag detection components, optical reader components (e.g., an optical sensor to detect one-dimensional bar codes such as Universal Product Code (UPC) bar code, multi-dimensional bar codes such as QR code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information can be derived via the communication components 836, such as location via Internet Protocol (IP) geolocation, location via Wi-Fi® signal triangulation, location via detecting an NFC beacon signal that may indicate a particular location, and so forth.

The various memories (i.e., 816, 818, 820, and/or memory of the processor(s) 804) and/or the storage unit 820 can store one or more sets of instructions 802 and data structures (e.g., software) embodying or utilized by any one or more of the methodologies or functions described herein. These instructions (e.g., the instructions 802), when executed by the processor(s) 804, cause various operations to implement the disclosed implementations.

As used herein, the terms “machine-storage medium,” “device-storage medium,” and “computer-storage medium” mean the same thing and can be used interchangeably. The terms refer to a single or multiple storage devices and/or media (e.g., a centralized or distributed database, and/or associated caches and servers) that store executable instructions 802 and/or data. The terms shall accordingly be taken to include, but not be limited to, solid-state memories, and optical and magnetic media, including memory internal or external to processors 804. Specific examples of machine-storage media, computer-storage media, and/or device-storage media include non-volatile memory, including by way of example semiconductor memory devices, e.g., erasable programmable read-only memory (EPROM), electrically erasable programmable read-only memory (EEPROM), field-programmable gate array (FPGA), and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 838 can be an ad hoc network, an intranet, an extranet, a virtual private network (VPN), a local-area network (LAN), a wireless LAN (WLAN), a wide-area network (WAN), a wireless WAN (WWAN), a metropolitan-area network (MAN), the Internet, a portion of the Internet, a portion of the public switched telephone network (PSTN), a plain old telephone service (POTS) network, a cellular telephone network, a wireless network, a Wi-Fi® network, another type of network, or a combination of two or more such networks. For example, the network 754 or a portion of the network 754 can include a wireless or cellular network, and the coupling 758 can be a Code Division Multiple Access (CDMA) connection, a Global System for Mobile communications (GSM) connection, or another type of cellular or wireless coupling. In this example, the coupling 756 can implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High-Speed Packet Access (HSPA), Worldwide Interoperability for Microwave Access (WiMAX), Long-Term Evolution (LTE) standard, others defined by various standard-setting organizations, other long-range protocols, or other data transfer technology.

The instructions 802 can be transmitted or received over the network 838 using a transmission medium via a network interface device (e.g., a network interface component included in the communication components 836) and utilizing any one of a number of well-known transfer protocols (e.g., Hypertext Transfer Protocol (HTTP)). Similarly, the instructions 802 can be transmitted or received using a transmission medium via the coupling 844 (e.g., a peer-to-peer coupling) to the devices 840. The terms “transmission medium” and “signal medium” mean the same thing and can be used interchangeably in this disclosure. The terms “transmission medium” and “signal medium” shall be taken to include any intangible medium that is capable of storing, encoding, or carrying the instructions 802 for execution by the machine 800, and include digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a manner as to encode information in the signal.

The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

Unless otherwise indicated, all numbers expressing quantities of physical properties, chemical properties, dimensions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the implementations described herein. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; #14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

### Example Aspects of the Disclosure

A numbered non-limiting list of aspects of the present subject matter is presented below.
Aspect 1. A method comprising: obtaining, by a computing system including one or more processors and memory, blood glucose data, the blood glucose data corresponding to blood glucose levels of a number of training subjects during one or more periods of time; obtaining, by the computing system, at least one of carbohydrate consumption data or insulin dose data for the number of training subjects, the carbohydrate consumption data indicating an amount of carbohydrates consumed by at least a portion of the number of training subjects during the one or more periods of time and the insulin dose data indicating a time at which at least a portion of the number of training subjects received a dose of insulin and a corresponding amount of insulin included in the dose; performing, by the computing system, a training process for a machine learning architecture that includes a convolutional neural network that implements one or more dilations, wherein the training process is performed using the blood glucose data and at least one of the carbohydrate consumption data or the insulin dose data to generate a trained global model to predict blood glucose measurements; obtaining, by the computing system, additional blood glucose data from an additional subject, the additional blood glucose data indicating one or more blood glucose measurements for the additional subject during a period of time; and determining, by the computing system and using the trained global model and the additional blood glucose data, a predicted blood glucose level of the additional subject at least 20 minutes after the period of time.
Aspect 2. The method of claim 1, comprising: performing, by the computing system, an additional training process for the machine learning architecture using the trained global model and the additional blood glucose data to generate a personalized model for the additional subject.
Aspect 3. The method of aspect 2, comprising: obtaining, by the computing system, additional carbohydrate consumption data for the additional subject, the additional carbohydrate consumption data indicating an amount of carbohydrate consumption that corresponds to a number of blood glucose levels for the additional subject; and wherein the additional training process uses the additional carbohydrate consumption data to generate the personalized model for the additional subject.
Aspect 4. The method of aspect 2, wherein: the trained global model includes a first number of components and a first number of weights that correspond to the first number of components; and the personalized model includes a second number of components and a second number of weights.
Aspect 5. The method of any one of aspects 1-4, wherein the machine learning architecture includes one or more artificial neural networks, and the method comprises: generating, by the computing system and using the convolutional neural network, intermediate data; providing, by the computing system, the intermediate data to the one or more artificial neural networks; and generating, by the computing system and using the one or more artificial neural networks, the predicted blood glucose level.
Aspect 6. The method of aspect 5, wherein: the machine learning architecture includes a first artificial neural network and a second artificial neural network.
Aspect 7. The method of aspect 6, comprising: providing, by the computing system, a first portion of the intermediate data to the first artificial neural network; and generating, by the computing system and using the first artificial neural network, a predicted mean value for a number of predicted values for the blood glucose level of the additional subject.
Aspect 8. The method of aspect 7, comprising: providing, by the computing system, a second portion of the intermediate data to the second artificial neural network; and generating, by the computing system and using the second artificial neural network, a standard deviation for the number of predicted values for the blood glucose level of the additional subject.
Aspect 9. The method of aspect 8, comprising: determining, by the computing system, the predicted blood glucose level of the additional subject based on the predicted mean value for the number of predicted values for the blood glucose level of the additional subject and based on the standard deviation of the number of predicted values for the blood glucose level of the additional subject.
Aspect 10. The method of any one of aspects 1-9, comprising determining, by the computing system, a recommendation for at least one of an amount of insulin to be taken by the additional subject or an amount of carbohydrates to be consumed by the additional subject based on the predicted blood glucose level of the additional subject.
Aspect 11. The method of aspect 10, comprising providing an insulin dose to the additional subject that corresponds to the amount of insulin indicated by the recommendation.
Aspect 12. The method of any one of aspects 1-11, comprising: determining, by the computing system, a recommendation indicating an amount of insulin to be taken by the additional subject based on the predicted blood glucose level of the additional subject; and causing, by the computing system, a notification to be accessible to the additional subject that includes the recommendation.
Aspect 13. The method of any one of aspects 1-12, comprising determining a recommendation indicating an amount of carbohydrates to be consumed by the additional subject based on the predicted blood glucose level of the additional subject; and causing a notification to be accessible to the additional subject that includes the recommendation.
Aspect 14. The method of any one of aspects 1-13, wherein: the additional blood glucose data is generated by a sensor that is located at least partially below an epidermis layer of the additional subject; the additional blood glucose data is received from at least one of a wearable device or a computing device of the additional subject; and an application executed by the computing device of the additional subject displays a recommendation indicating at least one of an amount of insulin to be taken by the additional subject or an amount of carbohydrates to be consumed by the additional subject, the recommendation being generated based on the additional blood glucose data.
Aspect 15. The method of any one of aspects 1-14, comprising: obtaining, by the computing system, physical activity data of the additional subject; determining, by the computing system and based on the additional blood glucose data, a recommendation indicating at least one of an amount of insulin to be taken by the additional subject or an amount of carbohydrates to be consumed by the additional subject; and modifying, by the computing system, the recommendation based on the physical activity data.
Aspect 16. The method of any one of aspects 1-15, wherein the additional blood glucose data includes a time series of blood glucose measurements with individual blood glucose measurements being taken a period of time after a previous individual blood glucose measurement.
Aspect 17. The method of any one of aspects 1-16, wherein the blood glucose data and the additional blood glucose data are obtained using a first set of computer-readable instructions that are stored in a first storage location and the first set of computer-readable instructions correspond to a first regulatory framework level; at least one of the carbohydrate consumption data for the number of training subjects or the insulin dose data for number of training subjects are obtained using the first set of computer-readable instructions; and the method comprises: determining, by the computing system and based on the additional blood glucose data, a recommendation indicating at least one of an amount of insulin to be taken by the additional subject or an amount of carbohydrates to be consumed by the additional subject, wherein the recommendation is determined using a second set of computer-readable instructions that are stored in a second storage location that is separate from the first storage location and the second computer-readable instructions correspond to a second regulatory framework level; and causing, by the computing system, a user interface to be displayed that includes the recommendation wherein the user interface is generated by the second set of computer-readable instructions.
Aspect 18. The method of aspect 17, wherein the second regulatory framework level includes a greater number of regulations than the first regulatory framework level.
Aspect 19. The method of any one of aspects 1-18, comprising: causing, by the computing system, one or more user interfaces to be displayed within an application executed by a computing device of the subject, the one or more user interfaces including one or more user interface elements to capture at least one of the carbohydrate consumption data, the insulin dose data, or physical activity data, wherein the one or more user interfaces are generated using the first set of computer-readable instructions.
Aspect 20. The method of any one of aspects 1-19, comprising: determining, by the computing system, an insulin to carbohydrate ratio that indicates an amount of insulin received by the subject in relation to an amount of carbohydrates consumed by the subject over a period of time, wherein the insulin to carbohydrate ratio is determined using the first set of computer-readable instructions; and obtaining, by the computing system, insulin sensitivity data that indicates an amount of change in blood glucose levels of the subject in response to one or more amounts of insulin, wherein the insulin sensitivity data is obtained using the first set of computer-readable instructions; and wherein a recommendation indicating at least one of an amount of insulin to be taken by the additional subject or an amount of carbohydrates to be consumed by the additional subject is determined based on the insulin to carbohydrate ratio and the insulin sensitivity data.
Aspect 21. The method of any one of aspects 1-20, comprising: determining, by the computing system, an amount of time that blood glucose levels of the additional subject have been within a target range of blood glucose levels; and causing, by the computing system, one or more user interfaces to be displayed indicating the amount of time that the blood glucose levels of the additional subject have been within a target range of blood glucose levels.
Aspect 22. The method of aspect 2, comprising performing, based on one or more criteria, an evaluation of the personalized model; determining, based on the evaluation, that predicted blood glucose levels generated by the personalized model have less than a threshold amount of variation over an additional period of time; and determining one or more additional predicted blood glucose levels using the personalized model; wherein the recommendation indicating at least one of an amount of insulin to be taken by the additional subject or an amount of carbohydrates to be consumed by the additional subject is based on the one or more additional predicted blood glucose levels.
Aspect 23. The method of aspect 22, comprising: causing, by the computing system, the personalized model to be stored in the first storage location.
Aspect 24. The method of aspect 2, comprising: performing, by the computing system and based on one or more criteria, an evaluation of the personalized model; determining, by the computing system and based on the evaluation, that predicted blood glucose levels generated by the personalized model have more than a threshold amount of variation over an additional period of time; and performing, based on the one or more criteria, an additional evaluation of the trained global model.
Aspect 25. The method of aspect 24, comprising: determining, by the computing system and based on the additional evaluation, that predicted blood glucose levels generated by the trained global model have less than the threshold amount of variation over the additional period of time; determining one or more additional predicted blood glucose levels using the trained global model; wherein the recommendation is based on the one or more additional predicted blood glucose levels.
Aspect 26. The method of aspect 24, comprising: determining, by the computing system and based on the additional evaluation, that predicted blood glucose levels generated by the trained global model have greater than the threshold amount of variation over the additional period of time; and causing a notification to be accessible to the subject indicating a level of variation in blood glucose levels predicted by the trained global model.
Aspect 27. A computing system comprising: one or more hardware processors; and memory storing computer-readable instructions that, when executed by the one or more hardware processors, perform operations comprising the method of any one of aspects 1-10, 12-26, and 51-53.
Aspect 28. A method comprising: obtaining, by a computing system including one or more processors and memory, blood glucose data, the blood glucose data corresponding to blood glucose levels of a subject, wherein the blood glucose data is obtained using a first set of computer-readable instructions that are stored in a first storage location and the first set of computer-readable instructions correspond to a first regulatory framework level; obtaining, by the computing system, at least one of carbohydrate consumption data for the subject or insulin dose data for the subject, the carbohydrate consumption data indicating an amount of carbohydrates consumed by the subject during one or more periods of time and the insulin dose data indicating a time at which the subject received a dose of insulin and an amount of insulin included in the dose, wherein at least one of the carbohydrate consumption data or the insulin dose data is obtained using the first set of computer-readable instructions; determining, by the computing system and based on the blood glucose data, a recommendation indicating at least one of an amount of insulin to be taken by a subject or an amount of carbohydrates to be consumed by the subject, wherein the recommendation is determined using a second set of computer-readable instructions that are stored in a second storage location that is separate from the first storage location and the second set of computer-readable instructions correspond to a second regulatory framework level; and causing, by the computing system, a user interface to be displayed that includes the recommendation wherein the user interface is generated by the second set of computer-readable instructions.
Aspect 29. The method of aspect 28, wherein the second regulatory framework level includes a greater number of regulations than the first regulatory framework level.
Aspect 30. The method of aspect 28 or 29, comprising: analyzing, by the computing system and using a machine learning architecture, the blood glucose data to determine a predicted blood glucose level of the subject at least 20 minutes after a period of time that corresponds to at least a portion of the blood glucose levels included in the blood glucose data, wherein a portion of the first set of computer-readable instructions is executed to implement the machine learning architecture; and wherein the recommendation is determined based on the predicted blood glucose level.
Aspect 31. The method of aspect 30, wherein: the machine learning architecture includes at least one convolutional neural network that implements one or more dilations and one or more additional artificial neural networks; the one or more additional artificial neural networks generate a mean predicted blood glucose level and a standard deviation that correspond to the mean predicted blood glucose level; and the predicted blood glucose level is determined based on the mean predicted blood glucose level and the standard deviation.
Aspect 32. The method of aspect 31, comprising: generating, by the computing system and using the at least one convolutional neural network, intermediate data; providing, by the computing system, the intermediate data to the one or more additional artificial neural networks; and generating, by the computing system and using the one or more additional artificial neural networks, the predicted blood glucose level.
Aspect 33. The method of aspect 32, wherein: the machine learning architecture includes a first artificial neural network and a second artificial neural network.
Aspect 34. The method of aspect 33, comprising: providing, by the computing system, a first portion of the intermediate data to the first artificial neural network; and generating, by the computing system and using the first artificial neural network, a predicted mean blood glucose level for a number of predicted values for the blood glucose level of the subject.
Aspect 35. The method of aspect 34, comprising: providing, by the computing system, a second portion of the intermediate data to the second artificial neural network; and generating, by the computing system and using the second artificial neural network, the standard deviation for the number of predicted values for the blood glucose level of the additional subject.
Aspect 36. The method of any one of aspects 28-35, comprising: obtaining, by the computing system, physical activity data for the subject, the physical activity data indicating an amount of activity performed by the subject during a period of time that corresponds to at least a portion of the blood glucose levels, wherein the physical activity data is obtained using the first set of computer-readable instructions; and wherein the recommendation is determined based on the physical activity data and the insulin consumption data.
Aspect 37. The method of aspect 36, comprising: causing, by the computing system, one or more user interfaces to be displayed within an application executed by a computing device of the subject, the one or more user interfaces including one or more user interface elements to capture at least one of the carbohydrate consumption data, the insulin dose data, or the physical activity data, wherein the one or more user interfaces are generated using the first set of computer-readable instructions.
Aspect 38. The method of any one of aspects 28-37, comprising: determining, by the computing system, an insulin to carbohydrate ratio that indicates an amount of insulin received by the subject in relation to an amount of carbohydrates consumed by the subject over a period of time, wherein the insulin to carbohydrate ratio is determined using the first set of computer-readable instructions; and obtaining, by the computing system, insulin sensitivity data that indicates an amount of change in blood glucose levels of the subject in response to one or more amounts of insulin, wherein the insulin sensitivity data is obtained using the first set of computer-readable instructions; and wherein the recommendation is determined based on the insulin to carbohydrate ratio and the insulin sensitivity data.
Aspect 39. The method of any one of aspects 28-38, comprising: determining, by the computing system, an amount of time that blood glucose levels of the subject have been within a target range of blood glucose levels; and causing, by the computing system, one or more user interfaces to be displayed indicating the amount of time that the blood glucose levels of the subject have been within a target range of blood glucose levels.
Aspect 40. The method of any one or aspects 28-39, comprising providing an insulin dose to the subject that corresponds to the amount of insulin indicated by the recommendation.
Aspect 41. The method of any one of aspects 28-40, comprising: obtaining, by the computing system, training data from a plurality of additional subjects, wherein the training data includes at least one of additional blood glucose levels of the plurality of additional subjects over a period of time or additional carbohydrate consumption data of at least a portion of the plurality of additional subjects; performing, by the computing system, a training process using the training data for a machine learning architecture to generate a trained global model that predicts blood glucose levels; and performing, by the computing system, an additional training process for the trained global model using the blood glucose data of the subject to generate a personalized model to predict blood glucose levels of the subject.
Aspect 42. The method of aspect 41, comprising: performing, by the computing system and based on one or more criteria, an evaluation of the personalized model; determining, by the computing system and based on the evaluation, that predicted blood glucose levels generated by the personalized model have less than a threshold amount of variation over an additional period of time; and determining, by the computing system, one or more additional predicted blood glucose levels using the personalized model; wherein the recommendation is based on the one or more additional predicted blood glucose levels.
Aspect 43. The method of aspect 42, comprising: causing, by the computing system, the personalized model to be stored in the first storage location.
Aspect 44. The method of aspect 41, comprising: performing, by the computing system and based on one or more criteria, an evaluation of the personalized model; and determining, by the computing system and based on the evaluation, that predicted blood glucose levels generated by the personalized model have more than a threshold amount of variation over an additional period of time; and performing, by the computing system and based on the one or more criteria, an additional evaluation of the trained global model.
Aspect 45. The method of aspect 44, comprising: determining, by the computing system and based on the additional evaluation, that predicted blood glucose levels generated by the trained global model have less than the threshold amount of variation over the additional period of time; determining, by the computing system, one or more additional predicted blood glucose levels using the trained global model; wherein the recommendation is based on the one or more additional predicted blood glucose levels.
Aspect 46. The method of aspect 44, comprising: determining, by the computing system and based on the additional evaluation, that predicted blood glucose levels generated by the trained global model have greater than the threshold amount of variation over the additional period of time; and causing, by the computing system, a notification to be accessible to the subject indicating a level of variation in blood glucose levels predicted by the trained global model.
Aspect 47. The method of any one of aspects 41-46, wherein: the trained global model includes a first number of components and a first number of weights that correspond to the first number of components; and the personalized model includes a second number of components and a second number of weights.
Aspect 48. The method of any one of aspects 28-47, wherein: the blood glucose data is generated by a sensor that is located at least partially below an epidermis layer of the subject; the blood glucose data is received from at least one of a wearable device or a computing device of the subject; and an application executed by the computing device of the subject displays the recommendation indicating at least one of the amount of insulin to be taken by the subject or the amount of carbohydrates to be consumed by the subject.
Aspect 49. The method of any one of aspects 28-48, wherein the blood glucose data includes a time series of blood glucose measurements with individual blood glucose measurements being taken a period of time after a previous individual blood glucose measurement.
Aspect 50. A computing system comprising: one or more hardware processors; and memory storing computer-readable instructions that, when executed by the one or more hardware processors, perform operations corresponding to the method of aspects 28-39, 41-49, and 54-56.
Aspect 51. The method of any one of aspects 1-26, wherein a recommendation user interface indicating a blood glucose modification recommendation is included in a series of user interfaces, the series of user interfaces including one or more data capture user interfaces and one or more review interfaces.
Aspect 52. The method of aspect 51, wherein the one or more data capture user interfaces includes at least one of a first data capture user interface to capture insulin dose information, a second data capture user interface to capture glucose level information, a third data capture user interface to capture carbohydrate consumption information, or a fourth data capture user interface to capture physical activity information.
Aspect 53. The method of aspect 52, wherein the first data capture user interface, the second data capture user interface, the third data capture user interface, and the fourth data capture user interface are displayed in order and the one or more review user interfaces are displayed in response to information being captured by each of the first data capture user interface, the second data capture user interface, the third data capture user interface, and the fourth data capture user interface.
Aspect 54. The method of any one of aspects 28-49, wherein a recommendation user interface indicating a blood glucose modification recommendation is included in a series of user interfaces, the series of user interfaces including one or more data capture user interfaces and one or more review interfaces.
Aspect 55. The method of aspect 54, wherein the one or more data capture user interfaces includes at least one of a first data capture user interface to capture insulin dose information, a second data capture user interface to capture glucose level information, a third data capture user interface to capture carbohydrate consumption information, or a fourth data capture user interface to capture physical activity information.
Aspect 56. The method of aspect 55, wherein the first data capture user interface, the second data capture user interface, the third data capture user interface, and the fourth data capture user interface are displayed in order and the one or more review user interfaces are displayed in response to information being captured by each of the first data capture user interface, the second data capture user interface, the third data capture user interface, and the fourth data capture user interface.
Aspect 57. A computing system comprising: one or more hardware processors; and memory storing computer-readable instructions that, when executed by the one or more hardware processors, cause the one or more hardware processors to perform operations comprising; obtaining blood glucose data, the blood glucose data corresponding to blood glucose levels of a number of training subjects during one or more periods of time; obtaining at least one of carbohydrate consumption data or insulin dose data for the number of training subjects, the carbohydrate consumption data indicating an amount of carbohydrates consumed by at least a portion of the number of training subjects during the one or more periods of time and the insulin dose data indicating a time at which at least a portion of the number of training subjects received a dose of insulin and a corresponding amount of insulin included in the dose; performing a training process for a machine learning architecture that includes a convolutional neural network that implements one or more dilations, wherein the training process is performed using the blood glucose data and at least one of the carbohydrate consumption data or the insulin dose data to generate a trained global model to predict blood glucose measurements; obtaining additional blood glucose data from an additional subject, the additional blood glucose data indicating one or more blood glucose measurements for the additional subject during a period of time; and determining using the trained global model and the additional blood glucose data, a predicted blood glucose level of the additional subject at least 20 minutes after the period of time.
Aspect 58. The computing system of aspect 57, wherein the memory stores additional computer-readable instructions that, when executed by the one or more hardware processors, cause the one or more hardware processors to perform additional operations comprising: determining a recommendation indicating an amount of insulin to be taken by the additional subject based on the predicted blood glucose level of the additional subject; and causing a notification to be accessible to the additional subject that includes the recommendation.
Aspect 59. The computing system of aspect 57 or 58, wherein the memory stores additional computer-readable instructions that, when executed by the one or more hardware processors, cause the one or more hardware processors to perform additional operations comprising: determining a recommendation indicating an amount of carbohydrates to be consumed by the additional subject based on the predicted blood glucose level of the additional subject; and causing a notification to be accessible to the additional subject that includes the recommendation.
Aspect 60. The computing system of any one of aspects 57-59, wherein: the additional blood glucose data is generated by a sensor that is located at least partially below an epidermis layer of the additional subject; the additional blood glucose data is received from at least one of a wearable device or a computing device of the additional subject; and an application executed by the computing device of the additional subject displays a recommendation indicating at least one of an amount of insulin to be taken by the additional subject or an amount of carbohydrates to be consumed by the additional subject, the recommendation being generated based on the additional blood glucose data.
Aspect 61. The computing system of any one of aspects 57-60, wherein the memory stores additional computer-readable instructions that, when executed by the one or more hardware processors, cause the one or more hardware processors to perform additional operations comprising: obtaining physical activity data of the additional subject; determining, based on the additional blood glucose data, a recommendation indicating at least one of an amount of insulin to be taken by the additional subject or an amount of carbohydrates to be consumed by the additional subject; and modifying the recommendation based on the physical activity data.
Aspect 62. The computing system of any one of aspects 57-61, wherein the additional blood glucose data includes a time series of blood glucose measurements with individual blood glucose measurements being taken a period of time after a previous individual blood glucose measurement.
Aspect 63. A method comprising: obtaining, by a computing system including one or more processors and memory, blood glucose data, the blood glucose data corresponding to blood glucose levels of a subject, wherein the blood glucose data is obtained using a first set of computer-readable instructions that are stored in a first storage location and the first set of computer-readable instructions correspond to a first regulatory framework level; obtaining, by the computing system, at least one of carbohydrate consumption data for the subject or insulin dose data for the subject, the carbohydrate consumption data indicating an amount of carbohydrates consumed by the subject during one or more periods of time and the insulin dose data indicating a time at which the subject received a dose of insulin and an amount of insulin included in the dose, wherein at least one of the carbohydrate consumption data or the insulin dose data is obtained using the first set of computer-readable instructions; determining, by the computing system and based on the blood glucose data, a recommendation indicating at least one of an amount of insulin to be taken by a subject or an amount of carbohydrates to be consumed by the subject, wherein the recommendation is determined using a second set of computer-readable instructions that are stored in a second storage location that is separate from the first storage location and the second set of computer-readable instructions correspond to a second regulatory framework level; and causing, by the computing system, a user interface to be displayed that includes the recommendation wherein the user interface is generated by the second set of computer-readable instructions.
Aspect 64. The method of aspect 63, wherein the second regulatory framework level includes a greater number of regulations than the first regulatory framework level.
Aspect 65. The method of aspect 63 or 64, comprising: analyzing, by the computing system and using a machine learning architecture, the blood glucose data to determine a predicted blood glucose level of the subject at least 20 minutes after a period of time that corresponds to at least a portion of the blood glucose levels included in the blood glucose data, wherein a portion of the first set of computer-readable instructions is executed to implement the machine learning architecture; and wherein the recommendation is determined based on the predicted blood glucose level.
Aspect 66. The method of aspect 65, wherein: the machine learning architecture includes at least one convolutional neural network that implements one or more dilations and one or more additional artificial neural networks; the one or more additional artificial neural networks generate a mean predicted blood glucose level and a standard deviation that correspond to the mean predicted blood glucose level; and the predicted blood glucose level is determined based on the mean predicted blood glucose level and the standard deviation.
Aspect 67. The method of any one of aspects 63-66, comprising: obtaining, by the computing system, physical activity data for the subject, the physical activity data indicating an amount of activity performed by the subject during a period of time that corresponds to at least a portion of the blood glucose levels, wherein the physical activity data is obtained using the first set of computer-readable instructions; and wherein the recommendation is determined based on the physical activity data and the insulin dose data.
Aspect 68. The method of aspect 67, comprising: causing, by the computing system, one or more user interfaces to be displayed within an application executed by a computing device of the subject, the one or more user interfaces including one or more user interface elements to capture at least one of the carbohydrate consumption data, the insulin dose data, or the physical activity data, wherein the one or more user interfaces are generated using the first set of computer-readable instructions.
Aspect 69. The method of any one of aspects 63-68, comprising: determining, by the computing system, an insulin to carbohydrate ratio that indicates an amount of insulin received by the subject in relation to an amount of carbohydrates consumed by the subject over a period of time, wherein the insulin to carbohydrate ratio is determined using the first set of computer-readable instructions; and obtaining, by the computing system, insulin sensitivity data that indicates an amount of change in blood glucose levels of the subject in response to one or more amounts of insulin, wherein the insulin sensitivity data is obtained using the first set of computer-readable instructions; and wherein the recommendation is determined based on the insulin to carbohydrate ratio and the insulin sensitivity data.
Aspect 70. The method of any one of aspects 63-69, comprising: determining, by the computing system, an amount of time that blood glucose levels of the subject have been within a target range of blood glucose levels; and causing, by the computing system, one or more user interfaces to be displayed indicating the amount of time that the blood glucose levels of the subject have been within a target range of blood glucose levels.
Aspect 71. The method of any one of aspects 63-70, comprising providing an insulin dose to the subject that corresponds to the amount of insulin indicated by the recommendation.
Aspect 72. A computing system comprising: one or more hardware processors; and memory storing computer-readable instructions that, when executed by the one or more hardware processors, cause the one or more hardware processors to perform operations comprising; obtaining blood glucose data, the blood glucose data corresponding to blood glucose levels of a subject, wherein the blood glucose data is obtained using a first set of computer-readable instructions that are stored in a first storage location and the first set of computer-readable instructions correspond to a first regulatory framework level; obtaining at least one of carbohydrate consumption data or insulin dose data for the subject, the carbohydrate consumption data indicating an amount of carbohydrates consumed by the subject during one or more periods of time and the insulin dose data indicating a time at which the subject received a dose of insulin and an amount of insulin included in the dose, wherein at least one of the carbohydrate consumption data of the insulin dose data is obtained using the first set of computer-readable instructions; determining, based on the blood glucose data, a recommendation indicating at least one of an amount of insulin to be taken by a subject or an amount of carbohydrates to be consumed by the subject, wherein the recommendation is determined using a second set of computer-readable instructions that are stored in a second storage location that is separate from the first storage location and the second set of computer-readable instructions correspond to a second regulatory framework level; and causing a user interface to be displayed that includes the recommendation wherein the user interface is generated by the second set of computer-readable instructions.
Aspect 73. The computing system of aspect 72, wherein the memory stores additional computer-readable instructions that, when executed by the one or more hardware processors, cause the one or more hardware processors to perform additional operations comprising: obtaining training data from a plurality of additional subjects, wherein the training data includes at least one of additional blood glucose levels of the plurality of additional subjects over a period of time or additional carbohydrate consumption data of at least a portion of the plurality of additional subjects; performing a training process using the training data for a machine learning architecture to generate a trained global model that predicts blood glucose levels; performing an additional training process for the trained global model using the blood glucose data of the subject to generate a personalized model to predict blood glucose levels of the subject.
Aspect 74. The computing system of aspect 73, wherein the memory stores additional computer-readable instructions that, when executed by the one or more hardware processors, cause the one or more hardware processors to perform additional operations comprising: performing, based on one or more criteria, an evaluation of the personalized model; determining, based on the evaluation, that predicted blood glucose levels generated by the personalized model have less than a threshold amount of variation over an additional period of time; and determining one or more additional predicted blood glucose levels using the personalized model; wherein the recommendation is based on the one or more additional predicted blood glucose levels.
Aspect 75. The computing system of aspect 74, wherein the memory stores additional computer-readable instructions that, when executed by the one or more hardware processors, cause the one or more hardware processors to perform additional operations comprising: causing the personalized model to be stored in the first storage location.
Aspect 76. The computing system of aspect 73, wherein the memory stores additional computer-readable instructions that, when executed by the one or more hardware processors, cause the one or more hardware processors to perform additional operations comprising: performing, based on one or more criteria, an evaluation of the personalized model; and determining, based on the evaluation, that predicted blood glucose levels generated by the personalized model have more than a threshold amount of variation over an additional period of time; and performing, based on the one or more criteria, an additional evaluation of the trained global model.
Aspect 77. The computing system of aspect 76, wherein the memory stores additional computer-readable instructions that, when executed by the one or more hardware processors, cause the one or more hardware processors to perform additional operations comprising: determining, based on the additional evaluation, that predicted blood glucose levels generated by the trained global model have less than the threshold amount of variation over the additional period of time; determining one or more additional predicted blood glucose levels using the trained global model; wherein the recommendation is based on the one or more additional predicted blood glucose levels.
Aspect 78. The computing system of aspect 76, wherein the memory stores additional computer-readable instructions that, when executed by the one or more hardware processors, cause the one or more hardware processors to perform additional operations comprising: determining, based on the additional evaluation, that predicted blood glucose levels generated by the trained global model have greater than the threshold amount of variation over the additional period of time; and causing a notification to be accessible to the subject indicating a level of variation in blood glucose levels predicted by the trained global model.

1-15. (canceled)

16. A method comprising:
causing, by one or more computing devices including one or more processors and memory, one or more first user interfaces to be displayed, the one or more first user interfaces including an insulin dose user interface that includes one or more first user interface elements to capture insulin dose information corresponding to an insulin dose administered to a subject and a time that the insulin dose was administered to the subject;
causing, by the one or more computing devices, one or more second user interfaces to be displayed, the one or more second user interfaces displaying information captured via one or more second user interface elements and including an additional user interface element that is selectable to request a blood glucose modification recommendation, wherein the one or more second user interface elements capture additional information included in calculating the blood glucose modification recommendation;
determining, by the one or more computing devices, that the insulin dose was administered to the subject within a threshold amount of time from a current time; and
causing, by the one or more computing devices, one or more third user interfaces to be displayed that include the blood glucose modification recommendation.

17. The method of claim 16, wherein the one or more second user interfaces include:
a glucose level user interface element that captures a blood glucose level of the subject;
a physical activity user interface element that captures physical activity information of the subject, the physical activity information indicating an amount of physical activity performed by the subject over a period of time; and
a carbohydrate consumption information user interface element that captures carbohydrate consumption information of the subject, the carbohydrate consumption information indicating an amount of carbohydrates consumed by the subject over an additional period of time.

18. The method of claim 17, wherein the blood glucose modification recommendation is determined in response to determining that the blood glucose level of the subject was captured within an additional threshold amount of time from the current time.

19. The method of claim 17, comprising:
determining, by the one or more computing devices, that an additional insulin dose was administered to the subject after the threshold period of time from an additional current time; and
causing, by the one or more computing devices, display of a notification for information related to a more recently administered insulin dose to be entered.

20. The method of claim 17, comprising:
determining, by the one or more computing devices, that the blood glucose level of the subject was obtained at greater than a threshold period of time from a current time; and
causing, by the one or more computing devices, display of a notification for a more recently obtained blood glucose level of the subject to be entered.

21. The method of claim 17, wherein the blood glucose modification recommendation is determined based on the insulin dose information corresponding to the subject, the blood glucose level of the subject, the physical activity information of the subject, and the carbohydrate consumption information of the subject.

22. The method of claim 16, wherein the one or more first user interfaces, the one or more second user interfaces, and the one or more third user interfaces comprise a series of user interfaces that are displayed in order.

23. The method of claim 22, wherein the one or more first user interfaces and the one or more second user interfaces are displayed in conjunction with a blood glucose modification recommendation session and the insulin dose information is unable to be modified during the blood glucose modification recommendation session.

24. The method of claim 16, wherein the one or more first user interfaces, the one or more second user interfaces, and the one or more third user interfaces are displayed by a first healthcare application and a second healthcare application integrated with the first healthcare application obtains blood glucose levels corresponding to the subject via one or more blood glucose sensors.

25. The method of claim 16, wherein the insulin dose information indicates an amount of insulin administered to the subject in conjunction with the insulin dose and a time that the insulin dose was administered to the subject.

26. The method of claim 16, wherein the blood glucose modification recommendation indicates at least one of an amount of insulin to be administered to the subject, an amount of carbohydrates to be consumed by the subject, an indication to refrain from physical activity for a period of time, an indication to alter an intensity of physical activity, an indication to contact a healthcare practitioner, or an indication regarding blood glucose levels of the subject being outside of a desired range of blood glucose levels.

27. The method of claim 16, comprising:
capturing, by the one or more computing devices using the one or more third user interfaces, input indicating one or more actions performed after the blood glucose modification recommendation was determined, wherein the one or more actions include at least one of an additional insulin dose being administered to the subject or consuming an amount of carbohydrates by the subject.

28. The method of claim 27, comprising:
causing, by the one or more computing devices, the one or more third user interfaces to display one or more differences between the blood glucose modification recommendation and the one or more actions performed after the blood glucose modification recommendation was determined.

29. The method of claim 16, comprising:
causing, by the one or more computing devices, one or more logbook user interfaces to be displayed, the one or more logbook user interfaces including user interface elements to capture input indicating information related to at least one of insulin doses administered to the subject, glucose levels of the subject, physical activity performed by the subject, or carbohydrates consumed by the subject.

30. The method of claim 29, comprising:
causing, by the one or more computing devices, one or more user interface elements of the one or more first user interfaces or the one or more second user interfaces to display information obtained via the one or more logbook user interfaces.

31. A system comprising:
one or more hardware processors; and
memory storing computer-readable instructions that, when executed by the one or more hardware processors, cause the one or more hardware processors to perform operations comprising:
causing one or more first user interfaces to be displayed, the one or more first user interfaces including an insulin dose user interface that includes one or more first user interface elements to capture insulin dose information corresponding to an insulin dose administered to a subject and a time that the insulin dose was administered to the subject;
causing one or more second user interfaces to be displayed, the one or more second user interfaces displaying information captured via one or more second user interface elements and including an additional user interface element that is selectable to request a blood glucose modification recommendation, wherein the one or more second user interface elements capture additional information included in calculating the blood glucose modification recommendation;
determining that the insulin dose was administered to the subject within a threshold amount of time from a current time; and
causing one or more third user interfaces to be displayed that include the blood glucose modification.

32. The system of claim 31, wherein:
the insulin dose information is obtained using a first set of computer-readable instructions that are stored in a first storage location and correspond to a first regulatory framework; and
the blood glucose modification recommendation is determined by executing a second set of computer-readable instructions that are stored in a second storage location separate from the first storage location, the second set of computer-readable instructions corresponding to a second regulatory framework.

33. The system of claim 32, wherein the one or more first user interfaces and the one or more second user interfaces are generated by executing at least a portion of the first set of computer-readable instructions.

34. The system of claim 32, wherein the memory stores one or more additional computer-readable instructions that, when executed by the one or more hardware processors, cause the one or more hardware processors to perform additional operations comprising:
determining an insulin to carbohydrate ratio that indicates an amount of insulin received by the subject in relation to an amount of carbohydrates consumed by the subject over a period of time, wherein the insulin to carbohydrate ratio is determined using the first set of computer-readable instructions; and
obtaining insulin sensitivity data that indicates an amount of change in blood glucose levels of the subject in response to one or more amounts of insulin, wherein the insulin sensitivity data is obtained using the first set of computer-readable instructions; and
wherein the blood glucose modification recommendation is determined based on the insulin to carbohydrate ratio and the insulin sensitivity data.

35. The system of claim 31, wherein:
the blood glucose modification recommendation is determined based on blood glucose data of the subject that is generated by a sensor that is located at least partially below an epidermis layer of the subject;
the blood glucose data is received from at least one of a wearable device or a computing device of the subject; and
an application executed by the computing device of the subject displays the one or more third user interfaces that include the blood glucose modification recommendation.
